(19) United States
(12) Patent Application Publication
(10) Pub. No.: US 2006/0002950 A1
(43) Pub. Date: Jan. 5, 2006

(54) Immunomodulatory protein derived from Trypanosomes and uses thereof

(76) Inventors: Patrick De Baetselier, Antwerpen (BE); Alain Beschin, Liernu (BE)

Correspondence Address: TRASK BRITT, P.O. BOX 2550, SALT LAKE CITY, UT, 84110, US

(21) Appl. No.: 11/159,902

(22) Filed: Jun. 23, 2005

Related U.S. Application Data
(63) Continuation of application No. PCT/EP03/51082, filed on Dec. 19, 2003.

(30) Foreign Application Priority Data
Dec. 23, 2002 (EP) 02080667.5

(51) Int. Cl. C12Q 1/68; G01N 33/53; G01N 33/569; C07H 21/04; C12N 1/10; A61K 39/00; C07K 14/44

(52) U.S. Cl. 424/185.1; 435/6; 435/7.22; 435/69.3; 435/320.1; 435/258.1; 530/350; 514/44; 536/23.7

(57) ABSTRACT

The present invention relates to the field of immunomodulation. More particularly, the present invention relates to the identification and isolation of a polypeptide derived from Trypanosomes that can be used to modulate the immune response in mammals.

### CROSS-REFERENCE TO RELATED APPLICATIONS

This application is a continuation of PCT International Patent Application No. PCT/EP2003/051082, filed on Dec. 19, 2003, designating the United States of America, and published, in English, as PCT International Publication No. WO 2004/056853 A2 on Jul. 8, 2004, which application claims priority to European Patent Application No. 02080667.5 filed Dec. 23, 2002, the entire contents of each are hereby incorporated herein by this reference.

### TECHNICAL FIELD

The present invention relates to the field of immunomodulation. More particularly, the present invention relates to the identification and isolation of a polypeptide derived from trypanosomes that can be used to modulate the immune response in mammals.

### BACKGROUND

African trypanosomes are extracellular protozoan parasites that inhabit the blood and tissues of their mammalian host. While Trypanosoma brucei rhodesiense and T b. gambiense are responsible for human sleeping sickness, T b. brucei, T. congolense, T vivax, T evansi, T equiperdum cause Nagana, a related disease in animals. African trypanosomes have developed highly sophisticated mechanisms to escape destruction by the host immune system by regularly changing their surface coat via a mechanism of antigenic variation (1) and by down-regulating the host immune system, leading to immunosuppression (2).

Immunosuppression was first discovered through the increased susceptibility of the host to secondary infections during African trypanosomiasis (3). The drastic suppression of the host immune system occurring in humans (4), cattle (5, 6), and murine models (7), was evidenced by a failure of T-cells from infected individuals to proliferate when exposed to specific or mitogen stimuli. Though different mechanisms were proposed, macrophages are key players in African trypanosome-elicited immunosuppression (8-12). The mechanisms through which macrophages exert their suppressive activity vary depending on the stage of infection and lymphoid tissue analyzed. During the early stage of infection, prostaglandins (PG) and nitric oxide (NO) impair mitogen-induced T-cell proliferation in the spleen, peritoneal cavity and lymph nodes of T. brucei-infected mice (13-16). At this stage, the role of the host cytokines IFN-γ and TNF-α in the inhibition of T-cell proliferative response seems limited to an up-regulation of PG and NO synthesis. During the late stage of infection, a NO-independent, IFN-γ-dependent mechanism of immunosuppression occurs in the lymph node compartment, while in the spleen, an IFN-γ-independent suppressive mechanism is elicited (16, 17).

Moreover, TNF-α was found to play an important role in the development of suppressive macrophages by inducing IFN-γ production in the lymph nodes of T. b. brucei-infected mice (18). In view of the relevance of immunosuppression in African trypanosomiasis, different attempts were performed to characterize the nature of T b. brucei product(s) responsible for the immunomodulatory activity. Initial studies have shown that high molecular weight complexes of at least four proteins isolated from a membrane trypanosome fraction exerted immunosuppressive activity in vivo (19, 20). Other investigators reported that suppressive activities were due to secreted trypanosome components (10). In this regard, Sternberg and Mabbott have shown that soluble bloodstream form components displayed macrophage-activating potential only in presence of IFN-γ, leading to NO-dependent suppression of T-cell proliferative response (21). Currently, few Trypanosoma immunomodulatory factors have been characterized: the T-lymphocyte triggering factor (TLTF) that induces CD8⁺ T-cells to secrete IFN-γ (22, 23); VSG, which is an important TNF-α-inducing factor through interaction with macrophages (24-26); and T b. brucei prostaglandin F_{2α} synthase that may account for the increased PG levels observed during trypanosomiasis (27). These immunomodulatory molecules may contribute to the pathogenesis of the disease. By using a macrophage cell line, we previously identified a T b. brucei fraction that induced the secretion of TNF-α and inhibited mitogen-induced T-cell proliferation (28). In the present invention, we disclose the isolated T b. brucei protein displaying similar immunomodulatory activities. We show that by interacting with macrophages this trypanosome factor (designated as TSIF, Trypanosome Suppressive Immunomodulating Factor) triggers type I cytokine and NO secretions, inhibits mitogen- and antigen-induced T-cell proliferation and impairs antigen-specific cytokine responses.

A second aspect of the invention deals with the diagnosis of African trypanosomiasis based on TSIF. African trypanosomiasis, the agent of sleeping sickness, is a disease of economic importance since it affects the health of both livestock and man and can be caused by infection with either of two subspecies of Trypanosoma brucei: T b. gambiense and T. b. rhodesiense. If untreated, the disease is fatal and, therefore, early diagnosis is essential. Moreover, as most of the drugs currently available for the treatment of sleeping sickness have serious side effects, demonstration of the parasite in tissue fluids of the patient before and also during follow-up of chemotherapeutic treatment is indicated. Several methods are currently used for diagnosis, being mainly clinical examination in combination with parasite and specific antibody detection.

Since parasite detection is often difficult to achieve due to low numbers of circulating parasites, indirect diagnostic methods have been developed of which only the antibody detection tests are currently being used with varying success (29-34). The most common test for serodiagnosis of T. b. gambiense sleeping sickness is the Card Agglutination Test for Trypanosomiasis (C.A.T.T.) (35). Despite the high sensitivity of the C.A.T.T. (approximately 90%), not all serologically positive cases can be confirmed parasitologically. Moreover, antibody detection tests cannot discriminate between current and cured infections and can be negative in case of low antibody titers, e.g., in early infections. Therefore, nucleic acid-based techniques could be advantageous as diagnostic tests due to their alleged sensitivity and specificity. Hitherto, PCR detection of African as well as American trypanosomes is based on the amplification of multi copy sequences to allow detection of trypanosomes, both from infected blood and tsetse flies. It has been shown that a PCR signal can be obtained from DNA of trypanosome extracts containing a single genome equivalent (36, 37). While these reports show that amplification of multiple copy sequences can be used to detect trypanosomes to a high degree of sensitivity, we have shown in the present invention that this test lacks specificity. We provide herein a diagnostic test based on the detection of the single copy gene sequence of TSIF for diagnosis of trypanosome infections.

### SUMMARY OF THE INVENTION

In the present invention, we have identified a molecule designated as trypanosome Suppressive Immunomodulatory Factor (TSIF). TSIF elicits suppressive macrophages in vivo and exhibits a suppressive activity on antigen- and mitogen-induced T-cell proliferation.

TSIF is also able, depending on the way of administration, to block the secretion of type I or type II cytokines induced by trypanosome unrelated antigens. The full-length TSIF cDNA with an open reading frame of 2499-bp has an ATG codon located 256-bp downstream of the mini-exon sequence and thus encodes a theoretical protein of 833 amino acids. The deduced protein with a molecular mass of 92-kDa has an isoelectric point of 4.75, 32 cysteine residues and five potential N-glycosylation sites. Hydropathy analysis suggests that the protein contains at least three highly hydrophobic regions with length to be membrane spanning. Furthermore, computational analysis suggests the presence of an internal signal sequence between amino acids 230-290. A Blast search with the deduced amino acid sequence of the newly identified protein revealed no homology with known proteins. Southern blot analysis of T. b. brucei DNA indicated a restriction pattern characteristic of a single copy gene, since digestion with restriction enzymes that cut once in the TSIF ORF generated fragments of dissimilar sizes.

Hybridization with genomic DNA from various species belonging to the genus Trypanosomatidae demonstrated the presence of the TSIF gene only in species from the subgenus Trypanosoma, suggesting no difference among the species regarding gene number and/or organization. Northern blot analysis revealed that TSIF was transcribed as a ˜2.9 kb mRNA in T. b. brucei bloodstream forms. The presence of a putative internal signal sequence between amino acids 230-290 may result in the synthesis of a mature TSIF with an approximate molecular mass of 70-kDa. Based on the above information, TSIF comprising the amino acids 280-833 and corresponding to the putative mature TSIF protein was engineered.

According to the present invention, there is provided an isolated polypeptide having the primary structural information of amino acids 1-833 as set forth in SEQ ID NO:2 or any functional fragment or allelic variant thereof possessing the biological property of having immunomodulating activity. According to another embodiment of the invention, there is provided an isolated polynucleotide (depicted in SEQ ID NO:1), referred to hereinbelow as TSIF (trypanosome suppressive immunomodulating factor), TSIF cDNA or TSIF gene, encoding a polypeptide having the biological property of having immunomodulating activity. According to another embodiment, there is provided a polypeptide having the primary structural information of amino acids 1-553 as set forth in SEQ ID NO:4 or any functional fragment or allelic variant thereof possessing the biological property of having immunomodulating activity.

According to another embodiment, there is provided a polynucleotide (depicted in SEQ ID NO:3) encoding a polypeptide having the biological property of having immunomodulating activity.

According to another embodiment, immunomodulating activity induced by TSIF or any functional fragment or allelic variant thereof is a reduction or suppression of the immune response which is at least 20%, preferably 30%, 40%, 50%, more preferably 60%, 70%, 80% or even more. Reduction or suppression of the immune response is reflected in the suppression of a Th1 immune response. Alternatively, it can be detected as a suppression of a Th2 immune response or it can even be detected as a suppression of a Th1 and a Th2 immune response. The specific or global suppression of a Th1 and/or a Th2 immune response can be measured by a reduction of cytokine production. Alternatively, a suppression of the immune response is measured as a reduction of T-cell proliferation, either an antigen-induced specific T-cell proliferation or a mitogen-induced T-cell proliferation.

According to another embodiment, a polypeptide is provided as described hereinabove wherein the polypeptide shares at least 40% homology, 50% homology, 60% homology, preferably at least 70% homology, more preferably at least 80% homology, most preferably at least 90% homology with SEQ ID NO:2 or SEQ ID NO:4.

According to yet another embodiment, a polynucleotide, encoding a polypeptide having immunomodulatory activity, is provided as described hereinabove wherein the polynucleotide shares at least 40% homology, 50% homology, 60% homology, preferably at least 70% homology, more preferably at least 80% homology, most preferably at least 90% homology with SEQ ID NO: 1 or SEQ ID NO:3.

Homology is determined using default parameters of a DNA sequence analysis software package developed by the Genetic Computer Group (GCG) at the University of Wisconsin.

As a number of terms and expressions are used throughout the detailed description to facilitate the understanding thereof, the following definitions are provided:

As used herein, the words “polynucleotide” may be interpreted to mean the DNA and cDNA sequence as detailed by Yoshikai et al. (1990) Gene 87:257, with or without a promoter DNA sequence as described by Salbaum et al. (1988) EMBO. J. 7(9):2807.

As used herein, “fragment” refers to a polypeptide or polynucleotide of at least about nine amino acids or 27 base pairs, typically 50 to 75, or more amino acids or base pairs, wherein the polypeptide contains an amino acid core sequence. A fragment may be, for example, a truncated TSIF isoform, modified TSIF isoform (as by amino acid substitutions, deletions, or additions outside of the core sequence), or other variant polypeptide sequence, but is not a naturally occurring TSIF isoform present in a trypanosome. If desired, the fragment may be fused at either terminus to additional amino acids or base pairs, which may number from 1 to 20, typically 50 to 100, but up to 250 to 500 or more. A “functional fragment” means a polypeptide fragment possessing the biological property of having immunomodulating activity or a polynucleotide fragment encoding immunomodulating activity.

According to further features in preferred embodiments of the invention described below, provided is a polynucleotide sequence that includes polynucleotide fragments encoding polypeptides having immunomodulating activity.

According to still further features in the described preferred embodiments, the polynucleotide fragment according to the present invention includes a portion (fragment) of SEQ ID NO:1 or SEQ ID NO:3 that encodes a polypeptide having immunomodulating activity.

According to still further features in the described preferred embodiments, the polypeptide encoded by the polynucleotide fragment includes an amino acid sequence as set forth in SEQ ID NO:2 or in SEQ ID NO:4 or a functional fragment thereof.

According to still further features in the described preferred embodiments, the polynucleotide fragment encodes a polypeptide having TSIF activity, which may therefore be allelic, subspecies and/or induced variant of the amino acid sequence set forth in SEQ ID NO:2 or SEQ ID NO:4. It is understood that any such variant may also be considered a homologue. The term “allelic variant” is used herein to denote any of two or more alternative forms of a gene occupying the same chromosomal locus. Allelic variation arises naturally through mutation and may result in phenotypic polymorphism within populations. Gene mutations can be silent (no change in the encoded polypeptide) or may encode polypeptides having altered amino acid sequence. The term “allelic variant” is also used herein to denote a protein encoded by an allelic variant of a gene.

According to still further features in the described preferred embodiments provided is a single-stranded polynucleotide fragment which includes a polynucleotide sequence complementary to at least a portion of a polynucleotide strand encoding a polypeptide having TSIF immunomodulating activity as described above.

According to still further features in the described preferred embodiments provided is a vector including a polynucleotide sequence encoding a polypeptide as depicted in SEQ ID NO:2 or SEQ ID NO:4 or any functional fragment thereof or allelic variant thereof possessing the biological property of having immunomodulating activity.

The vector may be of any suitable type including, but not limited to, a phage, virus, plasmid, phagemid, cosmid, bacmid or even an artificial chromosome. The polynucleotide sequence encoding a polypeptide having TSIF activity may include any of the above-described functional polynucleotide fragments or allelic variants thereof. As used herein, the term “regulatory element” refers to a genetic element which controls some aspect of the expression of nucleic acid sequences. For example, a promoter is a regulatory element that facilitates the initiation of transcription of an operably linked coding region. Other regulatory elements are splicing signals, polyadenylation signals, termination signals, etc. Transcriptional control signals in eucaryotes comprise “promoter” and “enhancer” elements. Promoters and enhancers consist of short arrays of DNA sequences that interact specifically with cellular proteins involved in transcription (T. Maniatis et al., Science 236:1237 (1987)). Promoter and enhancer elements have been isolated from a variety of eukaryotic sources including genes in yeast, insect and mammalian cells and viruses (analogous control elements, i.e., promoters, are also found in procaryotes). The selection of a particular promoter and enhancer depends on what cell type is to be used to express the protein of interest. Some eukaryotic promoters and enhancers have a broad host range while others are functional in a limited subset of cell types (for review, see S. D. Voss et al., Trends Biochem. Sci. 11:287 (1986), and T. Maniatis et al., supra (1987)).

The term “recombinant DNA vector” as used herein refers to DNA sequences containing a desired coding sequence and appropriate DNA sequences necessary for the expression of the operably linked coding sequence of TSIF or any functional or allelic variant thereof in a particular host organism (e.g., mammal). DNA sequences necessary for expression in procaryotes include a promoter, optionally an operator sequence, a ribosome binding site and possibly other sequences. Eukaryotic cells are known to utilize promoters, polyadenlyation signals and enhancers.

According to still further features in the described preferred embodiments, provided is a host cell that includes an exogenous polynucleotide fragment including a polynucleotide sequence encoding a polypeptide depicted in SEQ ID NO:2 or SEQ ID NO:4 or any functional fragment thereof or allelic variant thereof having immunomodulating activity.

The exogenous polynucleotide fragment may be any of the above-described fragments. The host cell may be of any type such as prokaryotic cell, eukaryotic cell, a cell line, or a cell as a portion of a multicellular organism (e.g., cells of a transgenic organism).

According to still further features in the described preferred embodiments provided is a recombinant protein including a polypeptide, TSIF, or a functional or allelic variant thereof having immunomodulating activity. The recombinant protein may be purified by any conventional protein purification procedure close to homogeneity and/or be mixed with additives. The recombinant protein may be manufactured using recombinant expression systems comprising bacterial cells, yeast cells, animal cells, insect cells, plant cells or transgenic animals or plants.

According to still further features in the described preferred embodiments provided is a pharmaceutical composition comprising, as an active ingredient, a recombinant polypeptide or an allelic fragment thereof or a functional fragment thereof having TSIF activity.

According to still further features in the described preferred embodiments provided is medical equipment comprising a medical device containing, as an active ingredient, a recombinant polypeptide or an allelic fragment thereof or a functional fragment thereof having TSIF activity.

According to still further features in the described preferred embodiments provided is a TSIF overexpression system comprising a cell overexpressing TSIF activity. The cell may be a host cell transiently or stably transfected or transformed with any suitable vector which includes a polynucleotide sequence encoding a polypeptide having TSIF activity and suitable promoter and enhancer sequences to direct expression of TSIF. However, the overexpressing cell may also be a product of an insertion (e.g., via homologous recombination) of a promoter and/or enhancer sequence downstream to the endogenous TSIF gene of the expressing cell, which will direct overexpression from the endogenous gene. The term “overexpression” as used herein refers to a level of expression that is higher than a basal level of expression typically characterizing a given cell under otherwise identical conditions.

In yet another embodiment, an isolated polypeptide having the primary structural information of amino acids as set forth in SEQ ID NO:2 or SEQ ID NO:4 or any functional fragment or allelic variant thereof possessing the biological property of having immunomodulating activity is used as a medicament.

In yet another embodiment, an isolated polynucleotide depicted in SEQ ID NO:1 and/or SEQ ID NO:3 or any functional fragment or allelic variant thereof possessing the biological property of having immunomodulating activity can be used as a medicament. [00361 In yet another embodiment, an isolated polypeptide as set forth in SEQ ID NO:2 or SEQ ID NO:4 or any functional fragment or allelic variant thereof possessing the biological property of having immunomodulating activity can be used for the preparation (or manufacture) of a medicament for the suppression of the immune response.

The wording “immunomodulating activity” should read as a reduction or a suppression of the immune response as described hereinbefore. Diseases, disorders or complications which have an over-reactivity of the immune system are of particular interest for treatment with TSIF. Such diseases comprise auto-immune disorders, rheumatic disorders, multiple sclerosis and the like. In a particular embodiment, complications where a graft-versus-host response is involved are of interest, such as, for example, transplant rejection.

The wording “Th1 immune response or a type 1 immune response” as well as “Th2 immune response or a type 2 immune response” is further clarified in the following. Upon T-Cell Receptor (TCR)—ligation, Th0 cells differentiate into distinct subsets characterized by their functions and cytokine production profiles. Thus, Th1 lymphocytes, characterized by the production of IL-2, IFN-γ and TNF-α contribute to cellular immunity whereas Th2 lymphocytes, mainly involved in humoral immunity, produce IL-4, IL-5, IL-13, IL-9 and IL-10. Numerous examples of the consequences on disease outcome of skewed Th1 to Th2 ratios have been reported in the art. Polarized Th2 responses have been implicated in pathological situations, such as Leishmania major, TBC, human leprosy, schistosomiasis and mycotic infections. The contribution of Th1 cells relative to Th2 cells to the developing autoimmune response determines for a large part whether or not this response leads to clinical disease. The chronic autoimmune graft-versus-host disease, which develops after the administration of mismatched lymphoid cells, can be prevented by switching a Th2 to a Th1 response through administration of IFN-γ at the time of cellular transfer. It is also described that the inefficiency of the immune response against a human glioma is caused by the presence of activated tumor-infiltrating lymphocytes, characterized by a predominant type 2 lymphokine production. These cytokines do not promote a tumoricidal immune response and, therefore, do not counteract the growth of the tumor. In allergic asthma, a predominant Th2 response has also been noted. There is increasing evidence that Th2 cell-derived cytokines are pivotal in the generation and persistence of asthma and lung inflammation. Finally, type II cytokine polarization have been involved in lung as well as liver fibrotic diseases.

The term “medicament to treat” relates to a composition comprising molecules as described above and a pharmaceutically acceptable carrier or excipient (both terms can be used interchangeably) to treat diseases as indicated above. Suitable carriers or excipients known to the skilled man are saline, Ringer's solution, dextrose solution, Hank's solution, fixed oils, ethyl oleate, 5% dextrose in saline, substances that enhance isotonicity and chemical stability, buffers and preservatives. Other suitable carriers include any carrier that does not itself induce the production of antibodies harmful to the individual receiving the composition such as proteins, polysaccharides, polylactic acids, polyglycolic acids, polymeric amino acids and amino acid copolymers. The “medicament” may be administered by any suitable method within the knowledge of the skilled man. The preferred route of administration is parenterally. In parental administration, the medicament of this invention will be formulated in a unit dosage injectable form such as a solution, suspension or emulsion, in association with the pharmaceutically acceptable excipients as defined above. However, the dosage and mode of administration will depend on the individual. Generally, the medicament is administered so that the protein, polypeptide, or peptide of the present invention is given at a dose between 1 μg/kg and 10 mg/kg, more preferably between 10 μg/kg and 5 mg/kg, most preferably between 0.1 and 2 mg/kg. Preferably, it is given as a bolus dose. Continuous infusion may also be used and includes continuous subcutaneous delivery via an osmotic minipump. If so, the medicament may be infused at a dose between 5 and 20 μg/kg/minute, more preferably between 7 and 15 μg/kg/minute.

In yet another embodiment, an isolated polynucleotide encoding TSIF or any functional fragment of the polynucleotide or allelic variant thereof possessing the biological property of encoding immunomodulating activity is used for the preparation of a medicament for the suppression of the immune response. Thus, in other words, the present invention provides nucleic acids of TSIF described herein for the transfection of cells in vitro and in vivo. These nucleic acids can be inserted into any of a number of well-known vectors for the transfection of target cells and organisms as described below. The nucleic acids are transfected into cells, ex vivo or in vivo, through the interaction of the vector and the target cell. The nucleic acids for TSIF, under the control of a promoter, then express TSIF or any functional fragment or allelic variant thereof of the present invention. Such gene therapy procedures have been used to correct acquired and inherited genetic defects, cancer, and viral infection in a number of contexts. The ability to express artificial genes in humans facilitates the prevention and/or cure of many important human diseases, including many diseases which are not amenable to treatment by other therapies (for a review of gene therapy procedures, see, Nabel and Feigner, TIBTECH 11:211-217 (1993); Mintani and Caskey, TIBTECH 11:162-166 (1993); Mulligan, Science 926-932 (1993); Dillon, TIBTECH 11:167-175 (1993); Van Brunt, Biotechnology 6(10):1149-1154 (1998); Vigne, Restorative Neurology and Neuroscience 8:35-36 (1995); Kremer and Perricaudet, British Medical Bulletin 51(1):31-44 (1995); Haddada et al., in Current Topics in Microbiology and Immunology (Doerfler and Böhm eds., 1995); and Yu et al., Gene Therapy 1: 13-26 (1994)). Delivery of the gene or genetic material into the cell is the first critical step in gene therapy treatment of disease. A large number of delivery methods are well known to those of skill in the art. Preferably, the nucleic acids are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. Methods of non-viral delivery of nucleic acids include lipofection, microinjection, biolistics, virosomes, liposomes, immunoliposomes, polycation or lipid: nucleic acid conjugates, naked DNA, artificial virions, and agent-enhanced uptake of DNA. Lipofection is described in, e.g., U.S. Pat. Nos. 5,049,386, 4,946,787, and 4,897,355 and lipofection reagents are sold commercially (e.g., Transfectam™ and Lipofectin™). Cationic and neutral lipids that are suitable for efficient receptor-recognition lipofection of polynucleotides include those of Flegner, WO 91/17424 and WO 91/16024. Delivery can be to cells (ex vivo administration) or target tissues (in vivo administration). The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); U.S. Pat. Nos. 4,186,183, 4,217,344, 4,235,871, 4,261,975, 4,485,054, 4,501,728, 4,774,085, 4,837,028, and 4,946,787). The use of RNA or DNA viral-based systems for the delivery of nucleic acids take advantage of highly evolved processes for targeting a virus to specific cells in the body and trafficking the viral payload to the nucleus.

Viral vectors can be administered directly to patients (in vivo) or they can be used to treat cells in vitro and the modified cells are administered to patients (ex vivo). Conventional viral-based systems for the delivery of nucleic acids could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long-term expression of the inserted transgene.

Additionally, high transduction efficiencies have been observed in many different cell types and target tissues. The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would, therefore, depend on the target tissue. Retroviral vectors are comprised on cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., J. Virol. 66:2731-2739 (1992); PCT/US94/05700. In applications where transient expression of the nucleic acid is preferred, adenoviral based systems are typically used. Adenoviral-based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., U.S. Pat. No. 4,797,368; WO 93/24641; Kotin, Human Gene Therapy 5:793-801 (1994); Muzyczka. Construction of recombinant AAV vectors is described in a number of publications, including U.S. Pat. No. 5,173,414; Hermonat and Muzyczka, Proc. Natl. Acad. Sci. U.S.A. 81:6466-6470 (1984); and Samulski et al., J. Virol. 63:03822-3828 (1989). In particular, at least six viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent. pLASN and MFG-S are examples of retroviral vectors that have been used in clinical trials (Dunbar et al., Blood 85:3048-305 (1995); Kohn et al., Nat. Med. 1:1017-102 (1995); Malech et al., Proc. Natl. Acad. Sci. U.S.A. 94/22 12133-12138 (1997)). Pa317/pLASN was the first therapeutic vector used in gene therapy trials (Blaese et al., Science 270:475-480 (1995)). Transduction efficiencies of 50% greater have been observed for MFG-S packaged vectors (Ellem et al. Immunol. Immunother. 44(1):10-20 (1997); Dranoff et al., Hum. Gene Ther. 1:111-2 (1997)). Recombinant adeno-associated virus vectors (rAAV) are promising alternative gene delivery systems based on the defective and non-pathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system (Wagner et al., Lancet 351:9117 1702-3 (1998). Replication-deficient recombinant adenoviral vectors (Ad) are predominantly used transient expression gene therapy because they can be produced at high titer and they readily infect a number of different cell types. Most adenovirus vectors are engineered such that a transgene replaced the Ad E1a, E1b, and E3 genes; subsequently, the replication defector vector is propagated in human 293 cells that supply deleted gene function in trans. Ad vectors can transduce multiple types of tissues in vivo, including nondividing, differentiated cells such as those found in the liver, kidney and muscle system tissues.

Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Sterman et al., Hum. Gene Ther. 9:7 1083-1089 (1998); Alvarez et al., Hum. Gene Ther. 5:597-613 (1997); Topf et al., Gene Ther. 5:507-513 (1998)). Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome that are required for packaging and integration into the host genome.

Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses' outer surface. The ligand is chosen to have affinity for a receptor known to be present on the cell type of interest. For example, Han et al., Proc. Natl. Acad. Sci. U.S.A. 92/9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells. Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector. Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., patient). Various cell types suitable for ex vivo transfection are well known to those of skill in the art (see, e.g., Freshney et al., Culture of Animal Cells, A Manual of Basic Technique (3^{rd} ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such as GM-CSF, IFN-γ and TNF-α are known (see Inaba et al., J. Exp. Med. 176:1693-1702 (1992)). Stem cells are isolated for transduction and differentiation using known methods. For example, stem cells are isolated from bone marrow cells by panning the bone marrow cells with antibodies which bind unwanted cells, such as CD4+ and CD8+ (T-cells), CD45+ (panb cells), GR-1 (granulocytes), and lad (differentiated antigen-presenting cells) (see Inaba et al., J. Exp. Med. 176:1693-1702 (1992)).

Vectors (e.g., retroviruses, adenoviruses, liposomes, etc.) containing therapeutic nucleic acids can also be administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art and, although more than one route can be used to administer a particular composition, a particular route can often provide a more immediate and more effective reaction than another route. Administration is by any of the routes normally used for introducing a molecule into ultimate contact with blood or tissue cells. The nucleic acids are administered in any suitable manner, preferably with pharmaceutically acceptable carriers. Suitable methods of administering such nucleic acids are available and well known to those of skill in the art and, although more than one route can be used to administer a particular composition, a particular route can often provide a more immediate and more effective reaction than another route.

In yet another embodiment, the invention provides a method for the preparation of a diagnostic assay to detect the presence of a Trypanosoma infection in a tissue sample of a mammal comprising detecting the presence of the polynucleotide or a fragment or allelic variant thereof according to claim 2 and/or a polypeptide or a fragment or allelic variant thereof according to claim 1 in a tissue sample of the mammal, wherein the presence of the polynucleotide and/or polypeptide is indicative for a Trypanosoma infection. In a particular embodiment, the mammal is a human and Trypanosoma is a Trypanosoma brucei rhodesiense or a Trypanosoma brucei gambiense and the presence of Trypanosoma is indicative for the presence of sleeping sickness in the human.

Thus, the TSIF gene and gene product, as well as other products derived thereof (e.g., probes, antibodies), can be useful in the diagnosis of the presence of T. brucei gambiense or T. brucei rhodesiense and more specifically, in the diagnosis of sleeping disease. Diagnosis of the presence of Trypanosoma brucei rhodesiense or Trypanosoma brucei gambiense (causative for sleeping disease) can be accomplished by methods based upon the nucleic acids (including genomic and mRNA/cDNA sequences), proteins, and/or antibodies. Preferably, the methods and products are based upon the TSIF gene, protein or antibodies against the TSIF protein. For brevity of exposition, but without limiting the scope of the invention, the following description will focus upon uses of the TSIF gene (depicted in SEQ ID NOS:1 or 3) and gene product (SEQ ID NOS:2 or 4). It will be understood, however, that fragments or other homologous sequences (allelic versions) from other possible subspecies of T. brucei gambiense or T brucei rhodesiense causing sleeping disease will be equivalent for these diagnostic purposes. As will be appreciated by one of ordinary skill in the art, the choice of diagnostic methods of the present invention will be influenced by the nature of the available biological samples to be tested and the nature of the information required. Samples to be diagnosed can comprise blood, plasma, cerebrospinal fluid and the like. Thus, assays based upon the isolation of nucleic acids from a sample may be the preferred methods for diagnostics of the presence of Trypanosoma infection in mammals and, more particularly, for the presence of Trypanosoma brucei rhodesiense or T brucei gambiense infection in humans. When the diagnostic assay is to be based upon nucleic acids from a sample, either mRNA or genomic DNA may be used. With either mRNA or DNA, standard methods well known in the art may be used to detect the presence of a particular sequence either in situ or in vitro (see, e.g. Sambrook et al., eds. (1989) Molecular Cloning: A Laboratory Manual, Cold Spring Harbor Press, Cold Spring Harbor, N.Y.). In a preferred embodiment of the invention, the starting nucleic acid represents a sample of DNA isolated from a human patient. DNA is extracted from the cell source or body fluid using any of the numerous methods that are standard in the art. It will be understood that the particular method used to extract DNA will be chosen as being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction and/or phenol extractions can be used to obtain nucleic acid from cells or tissues, e.g., blood. In a specific embodiment, the cells may be directly used without purification of the target nucleic acid. For example, the cells can be suspended in hypotonic buffer and heated to about 90° C.-100° C., until cell lysis and dispersion of intracellular components occur, generally about 1 to 15 minutes. After the heating step, the amplification reagents may be added directly to the lysed cells. This direct amplification method may, for example, be used on blood or plasma. The preferred amount of DNA to be extracted for analysis of genomic DNA is at least 5 μg or more.

In a particular embodiment, the starting nucleic acid is RNA obtained, e.g., from a sample or tissue. RNA can be obtained from a cell or tissue according to various methods known in the art and described, e.g., in Molecular Cloning: A Laboratory Manual, 2nd Ed., ed. by Sambrook, Fritsch and Maniatis (Cold Spring Harbor Laboratory Press: 1989). For in situ detection of a nucleic acid sequence of TSIF, a sample of tissue may be prepared by standard techniques and then contacted with a probe, preferably one which is labeled to facilitate detection, and an assay for nucleic acid hybridization is conducted under stringent conditions which permit hybridization only between the probe and highly or perfectly complementary sequences. In many applications, the nucleic acids are labeled with directly or indirectly detectable signals or means for amplifying a detectable signal. Examples include radiolabels, luminescent (e.g. fluorescent) tags, components of amplified tags such antigen-labeled antibody, biotin-avidin combinations, etc. The nucleic acids can be subject to purification, synthesis, modification, sequencing, recombination, incorporation into a variety of vectors, expression, transfectibn, administration or methods of use disclosed in standard manuals such as Molecular Cloning: A Laboratory Manual (2nd Ed., Sambrook, Fritsch and Maniatis, Cold Spring Harbor), Current Protocols in Molecular Biology (Eds. Ausubel, Brent, Kingston, More, Feidman, Smith and Stuhl, Greene Publ. Assoc., Wiley-Interscience, NY, N.Y., 1992), or that are otherwise known in the art. A significant advantage of the use of either DNA or mRNA is the ability to amplify the amount of genetic material using the polymerase chain reaction (PCR), either alone (with genomic DNA) or in combination with reverse transcription (with mRNA to produce cDNA).

Other nucleotide sequence amplification techniques may be used, such as ligation-mediated PCR, anchored PCR and enzymatic amplification as will be understood by those skilled in the art. DNA samples may also be amplified by PCR prior to treatment with the appropriate restriction enzyme and the fragments of different sizes are visualized, for example, under UV light in the presence of ethidium bromide, after gel electrophoresis. DNA fragments may also be visualized by methods in which the individual DNA samples are not immobilized on membranes. The probe and target sequences may be in solution or the probe sequence may be immobilized. Autoradiography, radioactive decay, spectrophotometry and fluorometry may also be used to identify specific individual genotypes. Fragments of TSIF are sufficiently long for use as specific hybridization probes for detecting endogenous TSIF. Preferred fragments are capable of hybridizing to the corresponding TSIF or TSIF allelic variant under stringency conditions characterized by a specific hybridization buffer. In any event, the fragments are necessarily of length sufficient to be unique for hybridizing to TSIF or the TSIF allelic variant, i.e., has a nucleotide sequence at least long enough to define a novel oligonucleotide, usually at least about 14, 16, 18, 20, 22, or 24 bp in length, though such fragments may be joined in sequence to other nucleotides which may be nucleotides which naturally flank the fragment. For example, where the subject nucleic acids are used as PCR primers or hybridization probes, the subject primer or probe comprises an oligonucleotide complementary to a strand of the mutant or rare allele of length sufficient to selectively hybridize with the mutant or rare allele. Generally, these primers and probes comprise at least 16 bp to 24 bp complementary to the mutant or rare allele and may be as large as is convenient for the hybridizations conditions.

The wording “stringent hybridization conditions” is a term of art understood by those of ordinary skill in the art. For any given nucleic acid sequence, stringent hybridization conditions are those conditions of temperature, chaotropic salts, pH and ionic strength that will permit hybridization of that nucleic acid sequence to its complementary sequence and not to substantially different sequences. The exact conditions which constitute “stringent” conditions depend upon the nature of the nucleic acid sequence, the length of the sequence, and the frequency of occurrence of subsets of that sequence within other non-identical sequences. By varying hybridization conditions from a level of stringency at which non-specific hybridization conditions occurs to a level at which only specific hybridization is observed, one of ordinary skill in the art can, without undue experimentation, determine conditions which will allow a given sequence to hybridize only with complementary sequences. Hybridization conditions, depending upon the length and commonality of a sequence, may include temperatures of 20° C.-65° C. and ionic strengths from 5× to 0.1×SSC. Highly stringent hybridization conditions may include temperatures as low as 40° C.-42° C. (when denaturants such as formamide are included) or up to 60° C.-65° C. in ionic strengths as low as 0.1×SSC. These ranges, however, are only illustrative and, depending upon the nature of the target sequence and possible future technological developments, may be more stringent than necessary.

When a diagnostic assay is based upon the detection of TSIF proteins, a variety of approaches are possible. In some preferred embodiments, protein-based diagnostics will employ the ability of antibodies to bind to TSIF proteins. In particular, an assay in which one or more monoclonal antibodies capable of binding to one or more TSIF epitopes may be employed. Alternatively, a polyclonal antibody capable of binding to TSIF is used. The levels of antibody-TSIF binding in a sample obtained from a test subject (visualized by, for example, radiolabeling, ELISA or chemilurminescence) may be compared to the levels of binding to a control sample free of sleeping disease. Such antibody diagnostics may also be used for in situ immunohistochemistry using biopsy samples of tissues obtained from patients or may be used with fluid samples obtained from patients.

### DESCRIPTION OF THE FIGURES

FIG. 1: Identification of T b. brucei immunomodulatory fraction. Panel A: T b. brucei proteins precipitated in a window of 35-55% ammonium sulfate were fractionated by HPLC and evaluated for their capacity to activate the 2C11-12 macrophage cell line for TNF-α secretion and for inhibition of Con A-induced T-cell proliferation. Percent suppression was established comparing proliferation in co-cultures containing HPLC fraction- or PBS-pulsed 2011-12. Co-cultures were performed at a ratio of 7% 2C11-12 to Con A-activated lymph node cells. Proliferation in co-cultures containing PBS-pulsed 2C11-12 cells was 145,000±18,200 cpm. Panel B: Peritoneal cells elicited by HPLC fraction 4 (HPLC-4-PECs) or PBS (control-PECs) were co-cultured at a ratio of 7% with Con A-activated lymph node cells (LNC) and proliferation was monitored. Note that the presence of control peritoneal cells in co-cultures did not influence the proliferation of lymph node cells. Data are representative of three independent experiments. a: significantly lower (p<0.05) as compared to co-cultures containing PBS-pulsed 2C11-12 cells.

FIG. 2: Nucleotide, amino acid sequence and hydropathicity plot of TSIF. FIG. 2A: Spliced leader and poly (A) tail are boxed. Five potential N-glycosylation sites (underlined) and the 32 cysteines (thick underlined) are indicated. Arrows show computer-predicted internal signal sequence. FIG. 2B: Major hydrophobic regions are indicated: amino acids 210-235, 260-290, 795-817, respectively.

FIG. 3: Characterization of TSIF gene and cellular localization of TSIF protein. Panel A: Southern blot analysis of the TSIF gene: genomic DNA (5 μg) from different parasite species were digested with PvuI (cutting after nucleotide 1507 in the ORF) and hybridized with the full TSIF gene. Panel B: Northern blot analysis of TSIF gene expression: total RNA (30 μg) from bloodstream and procyclic T. b. brucei parasites was separated by formaldehyde agarose gel electrophoresis arid hybridized with the full TSIF gene. Panel C: Localization of TSIF protein in bloodstream T. b. brucei: parasites were stained with FITC-labeled anti-C-TSIF mAb and visualized by microscope. Panel D: Immunoprecipitation of TSIF protein: bloodstream T. b. brucei were labeled with ¹²⁵I. Lysate was immunoprecipitated with anti-rC-TSIF mAb (1) or anti-VSG polyclonal Ab (2). Precipitated material was submitted to SDS-PAGE and revealed by autoradiography. Panel E: Immunoblot analysis of TSIF protein: total lysate from bloodstream T. b. brucei (1) and fractions from parasite lysate bound to DEAE-52 column eluted by a gradient of NaCl (2, 3) were separated on SDS-PAGE. After blotting, membranes were revealed using anti-rC-TSIF mAb. The consecutive fractions from DEAE-52 column that scored positive (#21, #22) are illustrated.

FIG. 4: Secretion of pro-inflammatory mediators by rTSIF-activated macrophages. Thioglycollate-induced peritoneal exudate cells were cultured for two days with rTSIF, rTSIF digested with pronase or rTSIF pre-incubated with polymixin B. In parallel, cells were activated with LPS pre-incubated or not with polymixin B. Culture supernatants were tested for cytokine and nitric oxide productions. Data are representative of three independent experiments. a: significantly higher (p<0.05) as compared to non-stimulated cells (control).

FIG. 5: In vivo induction of suppressive cells by rTSIF. Panel A: Proliferative response of Con A-activated lymph node cells (LNC) co-cultured with peritoneal cells from mice treated intraperitoneally with rTSIF (5 μg) (rTSIF-PECs), rTSIF pre-incubated with anti-HPLC fraction 4 Ab (rTSIF/anti-HPLC #4-PECs), PBS (control PECS) and PBS pre-incubated with anti-HPLC fraction 4 Ab (anti-HPLC # 4-PECs). Panel B: Proliferative response of Con A-activated LNC co-cultured with rTSIF-PECs and control PECs was evaluated in the presence of different potential inhibitors. Panel C: Using the same cell populations as in Panel B, cells were co-cultured in absence of inhibitors in Transwell plates. Co-cultures were performed at a ratio of 7% peritoneal to lymph node cells. Note that the presence of control peritoneal cells in co-cultures did not influence the proliferation of lymph node cells. Data are representative of three independent experiments. a: significantly lower (p<0.05) as compared to control PECs.

FIG. 6: Inhibition of antigen-induced type II cytokine secretion by rTSIF. OVA (50 μg) emulsified in CFA was injected intra foot pad in mice that were pre-treated one day before with rTSIF (5 μg) or with PBS (control). Seven days later, draining popliteal lymph node cells were re-stimulated in vitro with OVA (5 μg/ml) for three days. Culture supernatants were assayed for cytokines production. Data are representative of three independent experiments. a: significantly lower (p<0.05) as compared to control mice.

FIG. 7: Inhibition of antigen-induced type I cytokine secretion by TSIF DNA vaccination. C57B1/6 mice were co-immunized intramuscularly three times at two-week intervals with 100 μg of pSecTag and 100 μg pcDNA3.1 plasmids (both from Invitrogen) encoding TSIF and OVA genes, respectively. Two weeks after the last immunization, spleen cells were stimulated ex vivo with OVA (50 μg/ml). OVA-specific T-cell proliferation (³H-Thy incorporation), and cytokine secretion in cell culture supernatants were compared three days later in mice immunized with pcDNA3.1 plasmid encoding the OVA gene and empty pSecTag plasmid (OVA) or with pcDNA3.1 and pSecTag plasmids encoding OVA and TSIF genes, respectively (OVA/TSIF).

FIG. 8: Secretion of TNF-α and induction of suppressive macrophages by rTSIF peptides. Top panel: Thioglycollate-induced peritoneal exudate cells were cultured for one to three days with rTSIF, or rTSIF fragments corresponding to amino acids 266-533 (rC6) or 46-269 (rD5). Culture supernatants were tested for TNF-α productions and compared with PBS-treated cells (control). Bottom panel: Proliferative response of Con A-activated lymph node cells (LNC) co-cultured with 7% peritoneal cells from mice treated intraperitoneally with rTSIF (5 μg) (rTSIF-PECs), or with equimolar concentration or rC6 (rC6-PECs) or rD5 (rD5-PECs) was compared with the proliferation in co-cultures containing peritoneal cells from PBS-treated mice (control PECS).

### DETAILED DESCRIPTION OF THE INVENTION

### EXAMPLES

Isolation of a Trypanosoma brucei brucei Immunomodulatory Fraction

We previously reported that upon interaction with opsonized Trypanosoma brucei brucei parasites, the macrophage hybridoma cell line (2C11-12) exhibits a suppressive activity on Con A-induced lymph node cell proliferation. Additionally, soluble extracts of T b. brucei triggered the 2C11-12 cell line to secrete TNF-α and to exert suppressive activities (28).

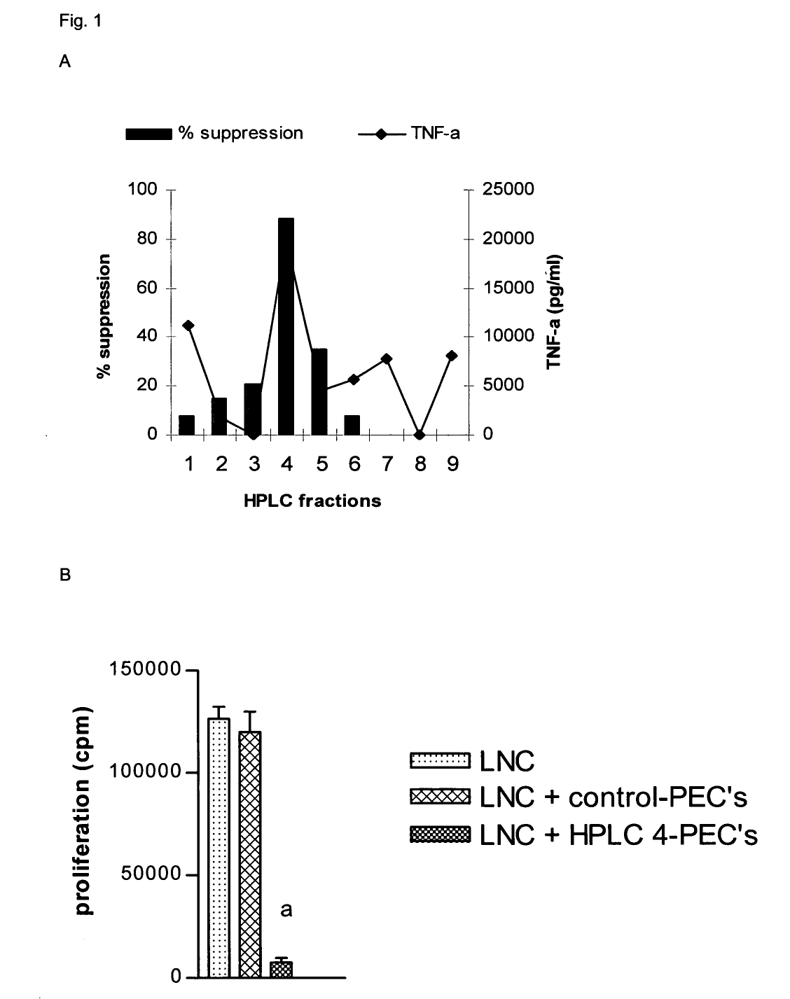

To identify molecule(s) responsible for these immunomodulatory activities, proteins present in T. b. brucei bloodstream lysate were fractionated by sequential ammonium sulfate precipitation. The presence of a macrophage-suppressive capacity was observed in the 35-55% ammonium sulfate fraction. This fraction was further fractionated by high performance liquid chromatography (HPLC). FIG. 1, Panel A, shows that mainly the HPLC fraction 4 from 35-55% ammonium sulfate precipitation triggered the 2C11-12 cell line to secrete TNF-α and to inhibit mitogen-induced T-cell proliferation. Both activities were abolished by treating the HPLC fraction 4 with pronase, indicating the involvement of protein(s) in the macrophage-activating capacity. Furthermore, this fraction elicited suppressive macrophages in vivo, since peritoneal macrophages from mice treated intraperitoneally (i.p.) with this fraction inhibited Con A-induced T-cell proliferation (FIG. 1, Panel B). Rabbit polyclonal antibodies generated against HPLC fraction 4 inhibited its suppressive activity.

Cloning, Molecular Characterization and Expression of TSIF Gene

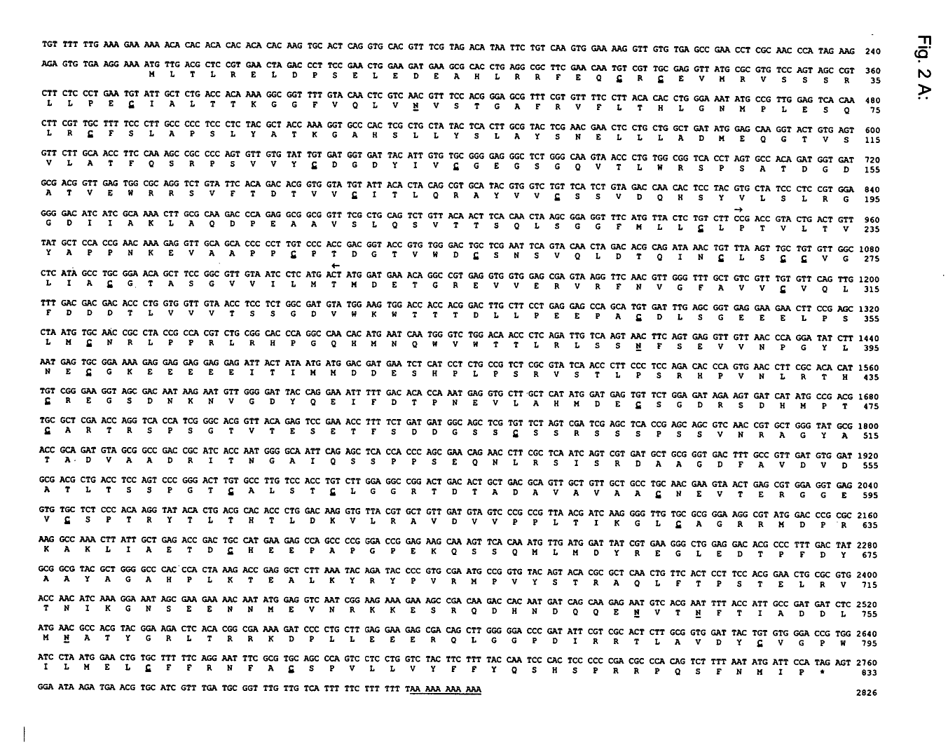

To identify gene(s) encoding the putative Trypanosoma immunomodulatory molecule(s), a bloodstream T. b. brucei cDNA expression library was screened with polyclonal anti-HPLC fraction 4 serum. From the eight positive phage clones isolated, five harbored sequences encoding for a T. b. brucei basal body component (38). The other three clones had no homology with sequences reported in data banks. cDNA from all clones were expressed as GST-fusion proteins and their capacity to elicit suppressive cells inhibiting Con A-induced T-cell proliferation was evaluated in vivo. The only clone exerting suppressive activity was selected for further research. The sequence of this clone revealed that the fragment of 1.65-kb represents the 3′ end of the gene. The missing 5′ end of this cDNA was amplified by RT-PCR using a gene-specific internal primer and a miniexon primer common to all trypanosome mRNA. The full-length cDNA with an open reading frame of 2499-bp had an ATG codon located 256-bp downstream of the miniexon sequence and thus encoded a theoretical protein of 833 amino acids (FIG. 2A). The deduced protein with a molecular mass of 92-kDa had an isoelectric point of 4.75, 32 cysteine residues and five potential N-glycosylation sites (FIG. 2A).

Hydropathy analysis suggested that the protein contained at least three highly hydrophobic regions with length to be membrane spanning (FIG. 2B). Furthermore computational analysis suggested the presence of an internal signal sequence between amino acids 230-290. A Blast search with the deduced amino acid sequence of the newly identified protein revealed no homology with known proteins. This protein was named TSIF (Trypanosoma Suppressive Immunomodulating Factor).

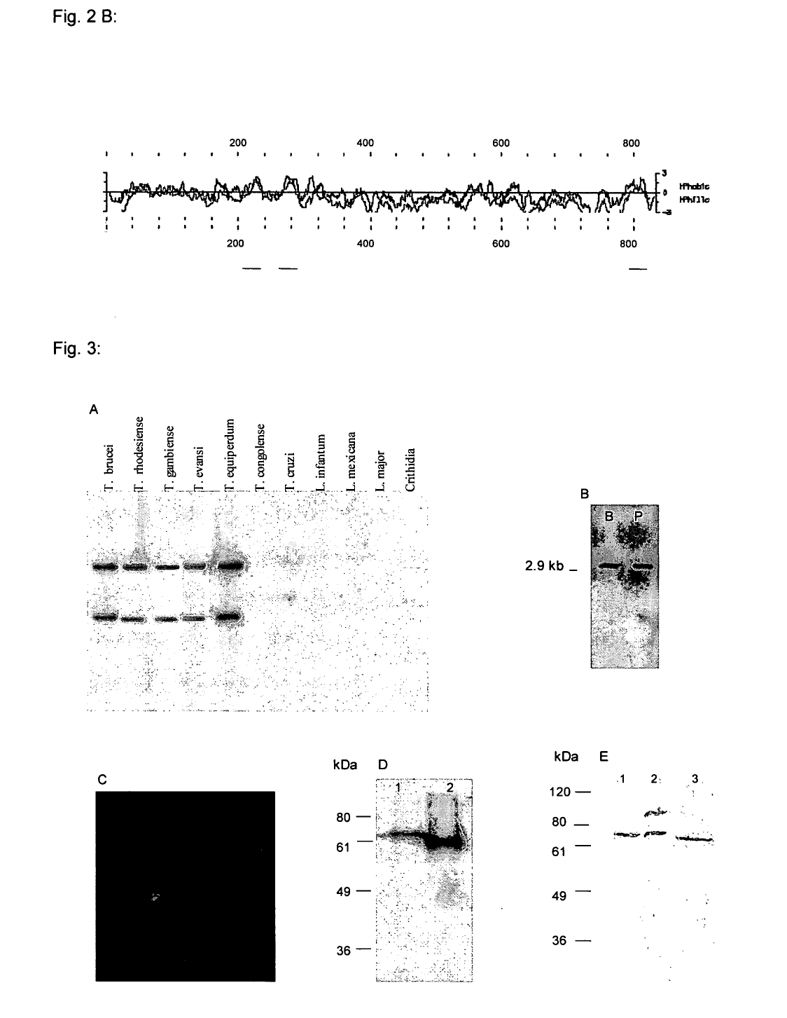

Southern blot analysis of T b. brucei DNA indicated a restriction pattern characteristic of a single copy gene, since digestion with restriction enzymes that cut once in the TSIF ORF generated fragments of dissimilar sizes. Hybridization with genomic DNA from various species belonging to the genus Trypanosomatidae demonstrated the presence of the TSIF gene only in species from the subgenus Trypanosoma, suggesting no difference among the species regarding gene number and/or organization. No hybridization signal was observed with T. cruzi, T. congolense, Leishmania or Crithidia parasites (FIG. 3, Panel A). Northern blot analysis revealed that TSIF was transcribed as a ˜2.9 kb mRNA in T b. brucei bloodstream and procyclic forms (FIG. 3, Panel B).

Expression of the complete TSIF ORF encountered difficulties. However, a protein containing an N-terminal histidine tag and encoding the C-terminal part of TSIF was generated (rC-TSIF, amino acids 543-833). A mAb elicited against rC-TSIF was used to analyze the cellular localization of native TSIF in the parasite. In immunofluorescence studies, this antibody showed uniform staining over the entire bloodstream T. b. brucei surface under conditions where the membrane was not permeabilized (FIG. 3, Panel C), suggesting that the native TSIF protein is located in the parasite membrane. These results were confirmed by surface-labeling immunoprecipitation. Indeed, following incubation of surface-labeled T. b. brucei bloodstream forms lysate with anti-rC-TSIF mAb, a protein showing an apparent molecular mass of 70-kDa was identified (FIG. 3, Panel D). Immunoblot analysis using anti-rC-TSIF mAb revealed a protein with a similar mass in total lysate from 10⁷ parasites (FIG. 3, Panel E). This size significantly differed from the 92-kDa molecular mass expected from the TSIF cDNA sequence. The likely interpretation of this discrepancy is that the 70-kDa component was processed from a 92-kDa precursor, as suggested by the detection of both components when trypanosome extracts (equivalent to 100 times more parasites) were bound to a DEAE column, then eluted by salt gradient (FIG. 3, Panel E). These data corroborate the computer prediction of a putative internal signal sequence between amino acids 230-290 that may result in the synthesis of a mature TSIF with an approximate molecular mass of 70-kDa.

Based on the above information, rTSIF comprising the amino acids 280-833 and corresponding to the putative mature TSIF protein was engineered, using the same procedure as for C-TSIF.

rTSIF Activates Mouse Macrophages

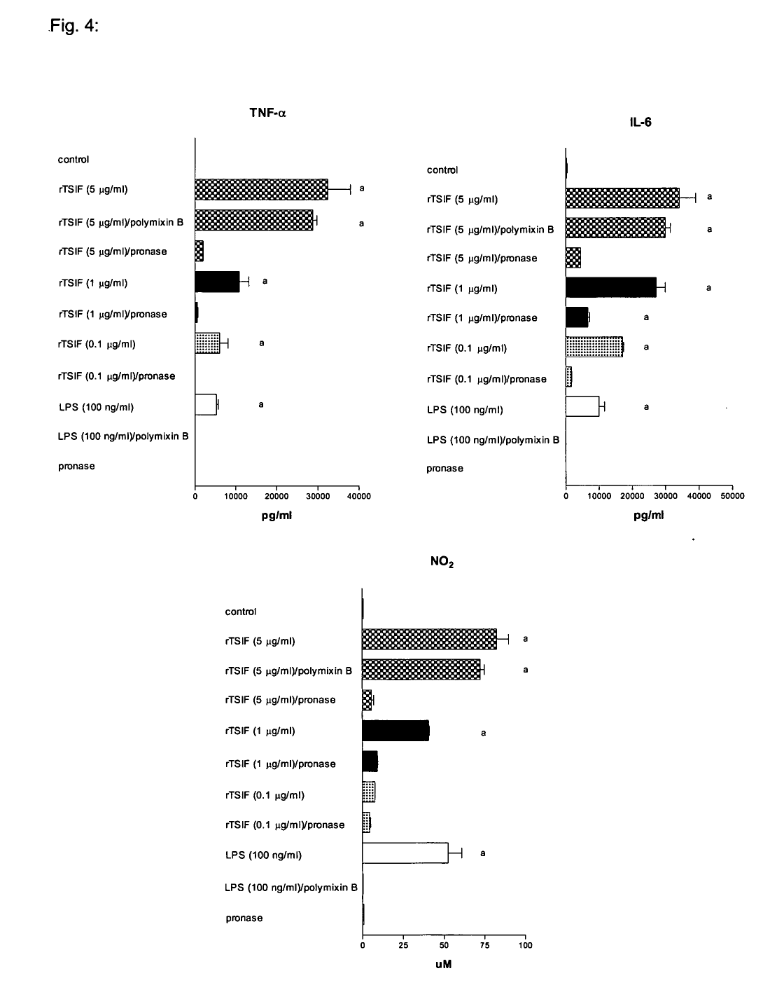

The macrophage-activating potential of rTSIF was tested on thioglycollate-elicited peritoneal macrophages, measuring cytokine and NO secretions into the cell culture medium. As shown in FIG. 4, rTSIF exerted high macrophage-activating activity as monitored by secretion of TNF-a and this activity was dose-dependent with a response starting at 0.1 μg/ml. Besides the production of the pro-inflammatory cytokine TNF-α, the rTSIF activated macrophages to produce high levels of the pleiotropic cytokine IL-6. The induction of these cytokines was paralleled by the up-regulation of NO in cell supernatants. Low levels of IL-10 were observed in the supernatants of rTSIF-activated macrophages (not shown). In vitro pre-activation of macrophages with IFN-γ induced higher secretions of cytokines and NO upon interaction with rTSIF. To ensure that the activation of macrophages did not result from LPS contamination, different controls were undertaken: (i) degradation of rTSIF with pronase eliminated its NO and cytokine-inciucing activity (FIG. 4); (ii) co-incubation with the LPS-inhibitor polymyxin B had no effect on the macrophage-activating potential of rTSIF (FIG. 4); (iii) the cytokine and NO-inducing activities of rTSIF were corroborated in the LPS-hyporesponsive C3H/HeJ mouse strain.

Collectively, these data indicate that rTSIF activates macrophages to secrete pro-inflammatory molecules.

rTSIF Elicits Suppressive Cells in vivo

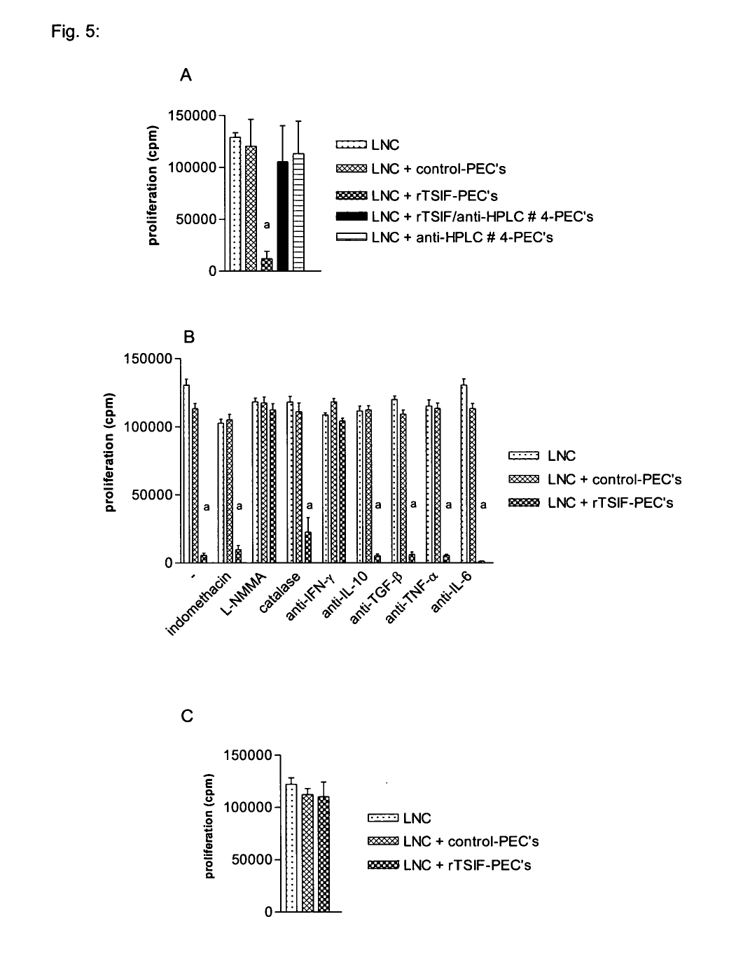

We investigated whether the strong macrophage-activating potential of rTSIF may trigger a suppressive state in vivo. Peritoneal cells from mice treated i.p. with rTSIF were co-cultured with Con A-activated lymph node cells and proliferation was monitored. Peritoneal cells from mice injected with rTSIF significantly suppressed T-cell proliferation (>90%) as compared with peritoneal cells from PBS-treated mice. Polyclonal antibodies elicited against HPLC fraction 4 and used to identify TSIF in T. b. brucei cDNA library inhibited the in vivo suppressive capacity of rTSIF (FIG. 5, Panel A). Additionally, rTSIF rendered the macrophage cell line 2C11-12 suppressive.

Previous studies showed macrophages inhibit T-cell activities via diverse mediators including prostaglandins, H₂O₂, NO and cytokines such as IFN-γ, TGF-β, IL-10 and TNF-α. The contribution of these mediators towards the suppressive activity of rTSIF-treated peritoneal cells was evaluated through addition of blocking agents, respectively, indomethacin, catalase, L-NMMA and neutralizing anti-IFN-γ, TGF-β, IL-10, and TNF-α antibodies. Neutralizing anti-IL-6 antibody was used as well in view of the induction of IL-6 secretion by rTSIF. Results indicate that suppression was mainly mediated by NO and IFN-γ, since the presence of the iNOS inhibitor L-NMMA and anti-IFN-γ antibodies in the co-cultures completely restored the proliferative capacity of Con A-activated lymph node cells. This was somehow surprising since similar levels of NO and IFN-γ were observed in co-cultures of lymph node cells and peritoneal cells from rTSIF-(36±10 μM NO₂ 7900±700 pg/ml IFN-γ) and PBS-treated mice (30±3 μM NO₂, 8500±500 μg/ml IFN-γ). We envisaged whether the rTSIF-elicited immunosuppressive activity involved a cell-cell contact. For that purpose, Con A-stimulated lymph node cells and peritoneal cells from mice treated i.p. with rTSIF were cultured in different compartments of Transwell plates. FIG. 5, Panel C, clearly indicates that suppression only occurred when suppressive cells were in contact with the mitogen-responding population. Thus, in addition to IFN-γ and NO, other signal(s) mediated by membrane-bound molecules are required to inhibit proliferation.

rTSIF Modulates Antigen-Specific Immune Responses

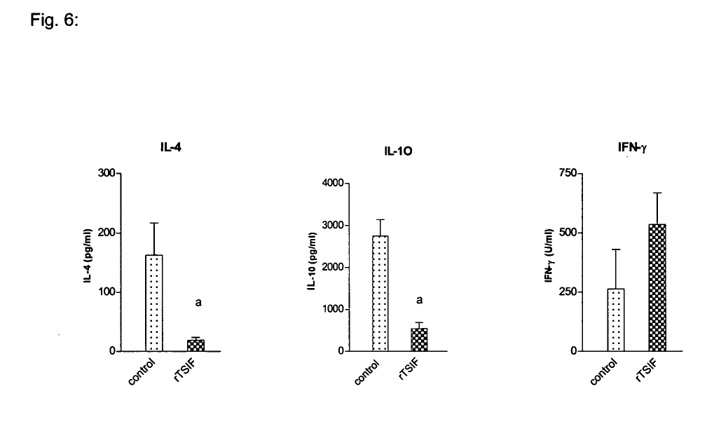

In view of the capacity of rTSIF to elicit suppressive cells in vivo, we subsequently evaluated the influence of rTSIF on antigen-specific immune responses. To this end, two approaches were undertaken. First, ovalbumin (OVA) was injected intra foot pad in mice that were pre-treated, either with rTSIF or with PBS (control). One week later, the draining popliteal lymph nodes were re-stimulated in vitro with OVA and productions of IL-4, IL-10 and IFN-γ were evaluated. As shown in FIG. 6, control mice immunized with OVA mounted a strong type II immune response characterized by secretions of IL-4 and IL-10, while OVA-immunized mice pre-treated with rTSIF exhibited significantly lower OVA-induced type II cytokine responses. The levels of the type I cytokine IFN-γ were lightly increased in rTSIF-pretreated mice. These results indicate that rTSIF modulates antigen-specific type II immune responses.

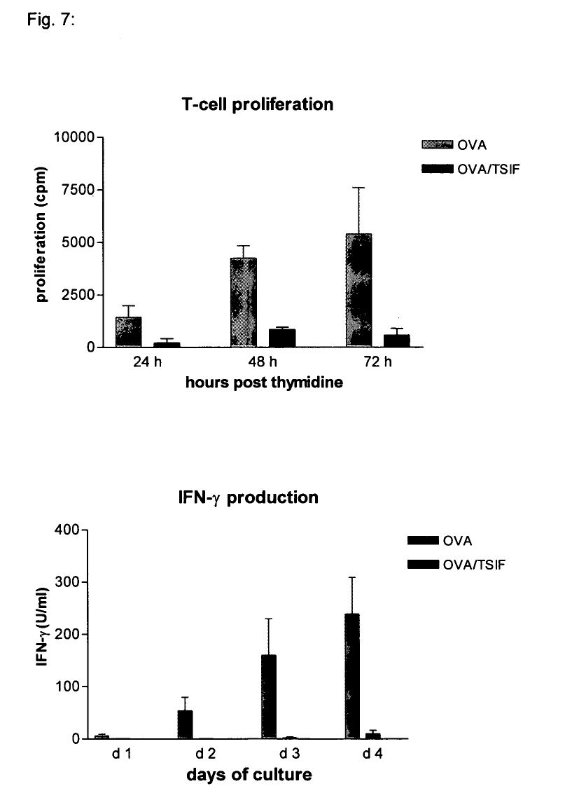

Second, mice were co-immunized intramuscularly with plasmids encoding OVA and TSIF genes. Two weeks after the last immunization, spleen cells were stimulated ex vivo with OVA. OVA-specific T-cell proliferation and cytokine secretion in cell culture supernatants were evaluated. Results indicate that TSIF DNA co-immunization suppressed OVA-specific T-cell proliferation (FIG. 7). Moreover, while spleen cells from mice immunized with OVA DNA secreted high levels of type I cytokine (IFN-γ) and marginal levels of type II cytokines (IL-4, IL-10), cells from mice co-immunized with OVA and TSIF DNA did not produce IFN-γ in response to OVA. The secretion of type II cytokines was not affected in OVA and TSIF DNA co-immunized animals.

Together, these data suggest that depending on the way of administration/delivery through the body, TSIF can suppress proliferation and type I or type II cytokine secretion by memory cells specific for trypanosome-unrelated antigens.

rTSIF Peptides Differentially Effect Cytokine Production and Suppressive Macrophages

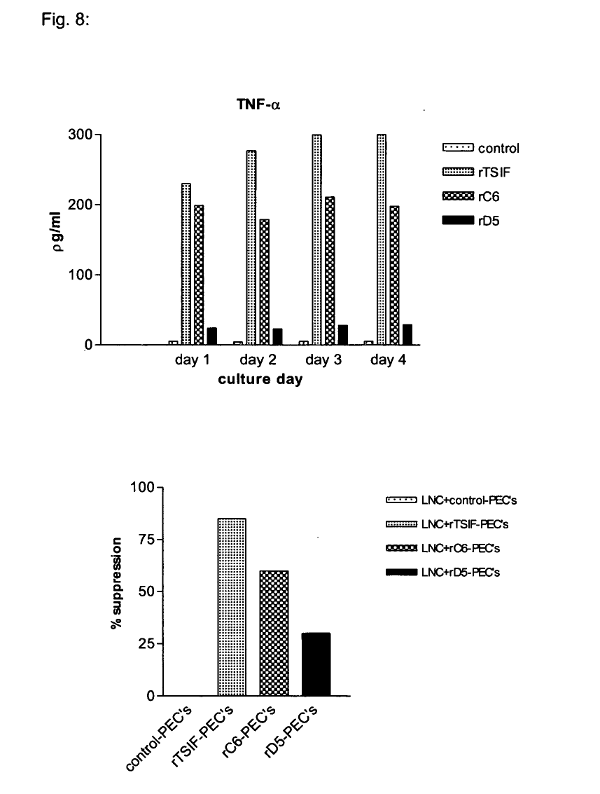

The ability of distinct rTSIF peptides to interact with macrophages was evaluated. To this end, peptides corresponding to the N-terminal (amino acid 46-269, rD5) or the C-terminal (amino acid 266-533, r06) part of rTSIF were generated. The macrophage-activating potential of these peptides was tested on thioglycollate-elicited peritoneal macrophages, measuring TNF-α secretion into the cell culture (FIG. 8, top panel). The C-terminal fragment rC6 induced the secretion of similar amounts of TNF-α as rTSIF, while the N-terminal fragment rD5 induced a marginal level of the cytokine. Moreover, peritoneal cells from mice injected with rC6 exerted a higher suppressive activity on Con A-induced lymph node cell proliferation than peritoneal cells from mice injected with rD5.

Together, these data suggest that distinct parts of the rTSIF protein differentially modulate the activity of immune cells. For instance, the TNF-inducing, as well as the suppression-inducing, potential of rTSIF mainly resides in the C-terminal part of the molecule.

PCR Amplification of the Single Copy TSIF Gene Sequence

To test the potential of the single copy TSIF gene as target for a sensitive diagnostic PCR assay, several pairs of primers were designed based on the TSIF sequence obtained from stock AnTat 1.1 T. brucei (J. Gomez, Department of Immunology, Parasitology and Ultrastructure, Flemish Interuniversity Institute for Biotechnology, Free University Brussels (VUB), Paardenstraat 65, B-1640 St-Genesius-Rode, Belgium). In order to optimize the assay in terms of sensitivity and specificity, parameters for both single and nested PCR assays were chosen, based on the length of the fragment to be amplified and the nature of the primers used. PCR amplification was optimized using extracted parasite DNA, followed by experiments on normal blood samples spiked with parasite DNA. The PCR assay was optimized for T. evansi strain ITMAS 110297.

In an initial experiment, a comparative PCR was performed on a dilution series of DNA samples, made to contain the equivalent of 0 up to 50,000 parasite genomes. A single PCR was performed using single pairs of primers (DTSIF A/S+DTSIF A/AS; DTSIF B/S+DTSIF B/AS; DTSIF C/S+DTSIF C/AS). In parallel, a nested PCR was performed using primer pair TSIF OP/S+TSIF OP/AS for the outer PCR reaction. The inner PCR was subsequently performed with either of the three above-mentioned primer pairs using 1/10^{th} of the first round PCR product as template. A single PCR resulted in amplification of the expected fragment until 5 pg purified parasite DNA was used per reaction. Nested PCR increased the sensitivity until 0.2 pg per reaction. This was for all three primer sets used. This is equivalent to a genome content of two parasites, assuming that one trypanosome parasite has a DNA content of 0.1 pg (39). The sensitivity of primer set DTSIF A tended to be slightly higher than that obtained with the two other primer sets since a weak, but clear, fragment was seen upon PCR amplification from 0.1 pg DNA. However, it cannot be ruled out that a certain ambiguity in the accuracy in serial dilutions in relation to exact numbers of parasites may account for the observed difference in sensitivity.

PCR Detection of T. evansi in Blood Samples Using TSIF and ESAG7 as Target

For PCR technology to be used in the diagnosis of trypanosomiasis, it must be adapted for the detection of parasites in samples of whole peripheral blood. To this end, EDTA-treated whole blood was spiked with different amounts of either purified trypanosomes or DNA purified using the commercially available QIAamp blood kit (Qiagen) or the SDS-proteinase K method. The TSIF-based nested PCR assay yielded a positive signal down to 50 trypanosomes/ml. However, using nested PCR to amplify conserved multicopy ESAG7 DNA target sequences could further increase the sensitivity to ten parasites/ml blood. Overall, the sensitivity of the PCR assay was similar for both DNA isolation methods used. Attempts to further increase the sensitivity using different sets of conditions were not successful.

Specificity of the Detection Method

In order to evaluate the specificity of the TSIF- and ESAG7-based PCR detection of Trypanosoma species, a library containing DNA from 37 representative trypanosome stocks was established. In addition, normal blood from mouse and human was tested as well as DNA from Leishmania species. No cross-reactions with DNA of normal blood samples from mouse or human were found upon single PCR amplification with primer sets DTSIF A and ESAG7, nor were there any cross-reactions with the Leishmania parasites. On the other hand, all trypanosomes belonging to the subgenus Trypanozoon produced a specific PCR band of the expected size when using the DTSIF A primer set, which could, therefore, be useful in the detection of these related parasites. The ESAG7 primer set scored positive for all trypanosomes belonging to the Trypanozoon subgenus and also for T. congolense TRT55, a stock belonging to the subgenus Dutonella.

Monitoring of Infection and of Treatment

In order to assess the efficacy of the TSIF A and ESAG7-based PCR for the clinical follow-up of patients or animals upon drug treatment, an experimental drug treatment protocol was set up. Female F1 mice were inoculated with 10⁴ T. b. brucei AnTat 1.1 ITMAS 241195, a pleiomorphic clone that produces chronic infections in mice. Treatment with DFMO was initiated three weeks after infection, the period involving cerebral trypanosomiasis, and administration of melarsoprol was initiated one week after DFMO treatment. A control group of five mice that did not receive any drug therapy following infection died of the disease 35-38 days post-infection.

Blood smears were scored positive for the presence of parasites starting at three days after experimental infection. In contrast, specific PCR products could be amplified as early as one day post-infection in blood samples and remained positive during infection although aparasitemic phases occurred. Indeed, parasitemia plunged below microscopical detection level during these phases whereas PCR signals remained positive. After three weeks of infection, parasites were also found in the brains, both by microscopic analysis and PCR detection. Moreover, BALB/c mice injected intraperitoneally with brain homogenates of the infected animals showed positive parasitemia after six days of injection, confirming cerebral disease. PCR and microscopic detection of parasites in the blood from all mice remained positive during the first two days of DMFO treatment. Subsequently, blood smears from some mice were negative by microscopic examination and eventually became all negative after eight days of treatment. During this period, all blood samples gave positive PCR signals when using both the TSIF A or ESAG primer sets. As soon as one day after treatment with melarsoprol, TSIF A-based PCR signals disappeared totally in blood, spleen, lymph nodes and brains. With the ESAG primer set, a positive signal was found in the blood and the brain of a mouse after one and two days of melarsoprol treatment, respectively. No trypanosome DNA amplification signal was demonstrated beyond this time point in any of the clinical samples collected until 60 days post-treatment. Also, the inoculation of homogenates of brain tissue taken 60 days after end of treatment failed to establish infection in susceptible mice, thus confirming successful elimination of the parasites. Clinical samples from control mice that only received therapy were also analyzed by TSIF A and ESAG-based PCR and remained negative throughout the whole treatment and follow-up period.

TSIF Modulates a Th2 Cytokine-Mediated Airway Inflammatory Response in Ovalbumin (OVA)-Sensitized Mice

Allergic asthma, a complex and chronic disease, is characterized by inflammation of the bronchial mucosa, involving activated eosinophils, mast cells, and CD4⁺ T-cells, as well as airway hyperresponsiveness, reversible bronchoconstriction, and elevated titers of circulating IgE. Observations from mouse models of allergic respiratory inflammation identified pulmonary cytokines characteristic of the Th2 subset of CD4⁺ T-cells, mainly IL-4, IL-5, IL-9, and IL-13, as crucial actors in the etiology of the human disease (Yssel and Groux, Int. Arch. Allergy Immunol. 2000, 121:10). Yet, the development of therapies targeted at neutralizing specific cytokines is hampered by intercytokine functional redundancy, thus limiting the effectiveness of the treatment (Riffo-Vasquez and Spina, Pharmacol. Ther. 2002, 94:185). Anti-IgE therapy has demonstrated limited clinical efficacy, exerting its action by reducing the amount of free IgE available to bind to effector cells (Busse et al., J. Allergy Clin. Immunol. 2001, 108:184). In this regard, modulating upstream processes, such as the preferential Th2 skewing in responses to inhaled allergen, may represent an alternative to prevent the development of the disease. The present example examines whether rTSIF alters the Th2 cell-mediated allergic asthma and reduces eosinophilic lung inflammation.

Recombinant proteins with N-terminal histidine tags corresponding to amino acids 280-833 (rTSIF) are prepared by using the pRSET/E. coli BL21 (DE3) system (Invitrogen). Transformed cells are induced for 5 hours in the presence of 1 mM (IPTG), then harvested and sonicated. Proteins present in inclusion bodies are dissolved in binding buffer (8 M urea-0.15 M NaCl-50 mM Tris-HCl pH 8.0) and purified by using Ni-nitrilotriacetic acid (NTA) resin (QIAGEN). Columns are washed with 50-column volumes of buffer containing decreasing concentrations of urea (8-6-2 M in 0.5 M NaCl-50 mM Tris-HCl pH 6.5). Bound proteins were eluted (0.5 M imidazole-2 M urea-0.1 M NaCl-50 mM Tris-HCl pH 6.5) and additionally purified by HPLC Superdex 75 column (Pharmacia) in PBS. Endotoxin contaminations, determined using Limulus amoebocyte lysate (LAL) assay (BioWhittaker), were below 20 units/mg protein. The amount of rTSIF produced as such is in the order of 1 to 5 mg and takes about two weeks.

Balb/c mice are sensitized by three intraperitoneal injections of 10 μg OVA adsorbed to 1 mg Al(OH)₃ on days 0, 7 and 14. On day 21, sensitized mice (five per experimental group) are challenged with 10 μg OVA admixed or not with 5 μg rTSIF in 30 μl PBS via the intranasal route. A third group of sensitized animals is treated with rTSIF one day before OVA challenge (i.e. at day 20). The fourth experimental group consists of sensitized animals treated with rTSIF at day 21 but not challenged with OVA. The last group consists of non-sensitized mice challenged with OVA. Bronchoalveolar ravages (BAL) are performed 48 hours after the last challenge and cytospin preparations are stained with May-Grünwald Giemsa as described in Sehra et al. (J. Immunol. 2003, 171:2080). Cytokine production is estimated by stimulating BAL cells (10⁶/ml, in triplicate) with 1 μg/ml anti-CD3 (clone 2C11) and 1 μg/ml anti-CD28 (clone 37.51) monoclonal antibody. Twenty-four hours later, IL-4, IL-5, IL-13, IL-10, IL-12 p70, TNF and IFN-γ are determined by cytokine-specific ELISA.

### Materials and Methods

Parasites

Trypanosome bloodstream forms were cultivated in rats or mice starting from a cryostabilate kept on liquid nitrogen. Trypanosoma theileri was cultured in vitro as described by Verloo et al. (40). Blood specimens were collected by heart puncture on EDTA. Visual inspection of parasites in the blood samples was performed by microscopic observation of at least 20 fields at 400× magnification. The parasitemia was estimated by the matching method (41).

Parasite Purification

For the optimization of the PCR-based diagnosis, we used T. evansi AnTat 3.1 ITMAS 270274C, a pleiomorphic clone kindly provided by N. Van Meirvenne (Prince Leopold Institute of Tropical Medicine, Antwerp, Belgium). This strain had been derived from trypanosomes isolated from blood of the Hydrochoerus hydrochoeris in South America in 1969 and produces a chronic infection in mice. The organism was maintained by serial passages in mice. Trypanosomes were purified from infected mouse blood by DEAE-52 anion-exchange column chromatography using phosphate-saline-glucose (PSG) solution as eluting buffer (42). Purified trypanosomes were kept at −80° C.

DNA Purification

DNA from trypanosome cells was purified by the sodium dodecyl sulphate (SDS)-proteinase K method (Gross-Bellard et al., 1973TO FIND=43). Serial dilutions were prepared in 10 mMTris-HCl, pH 8.0 and used as template for PCR amplification.

DNA from total blood treated with EDTA was isolated using two methods:
SDS-proteinase K lysis method: DNA from 100 μl fresh total blood was extracted in 500 μl blood lysis buffer (10 mM Tris-HCl pH 7.6, 10 mM EDTA, 0.1 M NaCl, 0.5% SDS, 300 μg/ml proteinase K) for 2 hours at 55° C. Samples were subsequently extracted twice with a mixture of phenol, chloroform, and isoamylalcohol (25:24:1). Sodium acetate was added to give a final concentration of 0.3 M and the nucleic acids were precipitated with two volumes of ethanol. After centrifugation for 15 minutes at 12,000×g, the DNA pellets were rinsed with 70% ethanol and dissolved in 100 μl sterile water.
QIAamp blood kit (Qiagen): this nucleic acid preparation kit combines the selective binding properties of a silica gel-based membrane with the speed of microspin technology. Blood samples were treated freshly or after preservation by mixing with an equal volume of stabilizing buffer AS-1 reagent (Qiagen). This procedure allows storage of blood samples for up to 12 weeks at temperatures up to 37° C. DNA was extracted from 200 μl blood according to the manufacturer's recommendations.
   Polymerase Chain Reaction

Primer pair TSIF OP/S (5′-CAGTAGCCGTCTTCTCCCTGAATG-3′)+TSIF OP/AS (5′-ATGTTGGTCACGCGCAGTTCCGTG-3′) was used in the outer PCR, yielding a PCR product of 2.1 kb. Primer pairs DTSIF A/S (5′-GTGAGTGTTCTTGCAACCTT CC-3′)+DTSIF A/AS (5′-GAAGTTGTAACAGACTGCAGCG-3′), DTSIF B/S (5′-GTTCTAGTCGATCGAGCTCA CC-3′)+DTSIF B/AS (5′-AGGGTGTGCGTCAGTGTATACC-3′) and DTSIF C/S (5′-AGGTATACACTGACGCACACCC-3′)+DTSIF C/AS (5′-TAAGAGCCTCGGTCTTTAG TGG-3′) were used for inner PCR, yielding PCR products of 314 bp, 328 bp and 268 bp, respectively.

ESAG7 primers were designed from the known sequence of T. brucei rhodesiense cDNA, encoding the transferrin-binding protein (23). Primer pair ESAG7 OP/S (5′-GAGGTTTTGGTTTGTGTTGTTG-3′)+ESAG7 OP/AS (5′-AGTATAGTTGAATTCGCTTTTAC-3′) was used in the outer PCR, yielding a PCR product of 1.3 kb. Primer pair ESAG7/S (5′-ACATTCCAGCAGGAGTTGGAG-3′)+ESAG7/AS (5′-CACGTGAATCCTCAATTTTGT-3′) was used for the inner PCR, yielding a PCR product of 238 bp.

Initially, the PCR conditions were optimized on serial dilutions of purified parasite DNA. DNA samples were amplified in a reaction mixture containing 1× PCR buffer (20 mM Tris-HCl pH 8.4, 50 mM KCl), 1.5 mM MgCl₂, 200 μM each of the four dNTPs, 0.5 μM each of the primers and 1.25 units of Platinum™ Taq DNA polymerase (Gibco BRL Life Technologies, Merelbeke, Belgium). Outer PCR reactions were performed on 10 μl template and 5 μl outer PCR product was subsequently used as template in the inner PCR reaction. Five μl of distilled water was added to PCR buffer as a negative amplification control.

For TSIF-based amplification, samples were incubated at 94° C. for 3 minutes as initial denaturation step, followed by 40 cycles of 1 minute at 94° C., 45 seconds at 60° C. and 1 minute 30 seconds at 72° C., and a final extension at 72° C. for 5 minutes. Nested PCR amplification was performed by an initial denaturation at 94° C. for 3 minutes, followed by 35 cycles of 1 minute at 94° C., 45 seconds at 60° C., and 1 minute at 72° C., and a final extension at 72° C. for 5 minutes.

Outer PCR for ESAG7 was performed using the same conditions as for TSIF-based amplification with the exception that the annealing of the primers was performed at 55° C. A 15-μl sample of each PCR product was finally tested by agarose gel electrophoresis.

Experimental Infection and Therapeutic Intervention

Experimental infections in mice were performed with T. b. brucei AnTat 1.1E ITMAS 241195 kindly provided by Dr. N. Van Meirvenne (ITG, Belgium). This pleiomorphic clone has been derived from trypanosomes isolated from blood of the Tragelaphus scriptus in Uganda in 1966 and produces a chronic infection in mice, allowing them to survive for at least 40 days if untreated.

Female F1 mice were inoculated intraperitoneally with 10⁴ T. b. brucei AnTat 1.1E parasites and blood parasitemia was followed by microscopic analysis and PCR. Three weeks after infection, when the central nervous system became infected and blood parasitemia reached about 10⁶ parasites/ml blood, the animals were treated for 15 consecutive days with 2% DFMO in the drinking water. Mice consumed an average of 5 ml of 2% drug solution/animal/day yielding a dose rate of 3 mg/g of body weight/day. One week after initiation of DFMO treatment, melarsoprol was administered intravenously for four consecutive days at a dose of 14.4 mg melarsoprol/kg body weight. Great care was taken to ensure no perivenous drug leakage to avoid phlebitis. Blood samples were taken throughout the infection and after treatment for microscopy and PCR. During therapy and at the end of the follow-up period, mice were sacrificed and blood, spleen, lymph nodes and brain samples were removed and processed for PCR analysis or injection into mice.

### REFERENCES

1. Pays E., S. Lips, D. Nolan, L. Vanhamme and D. Perez-Morga. 2001. The VSG expression sites of Trypanosoma brucei: multipurpose tools for the adaptation of the parasite to mammalian hosts. Mol. Biochem. Parasitol. 114:1-16.
2. Sileghem M., J. Flynn, A. Dali, P. De Baetselier and J. Naessens. 1994. African Trypanosomiasis. In Parasitic Infections and the Immune System, 1-51.
3. Castellani A. 1903. Report of the Sleeping Sickness Commission. Proc. R. Soc. London 1.
4. Greenwood B. M., H. C. Whittle and D. H. Molyneux. 1973. Immunosuppression in Gambian trypanosomiasis. Trans. R. Soc. Trop. Med. Hyg. 67:846-850.
5. Flynn J. N. and M. Sileghem. 1991. The role of the macrophage in induction of immunosuppression in Trypanosoma congolense-infected cattle. Immunology 74:310-316.
6. Taylor K. A. 1998. Immune responses of cattle to African trypanosomes: protective or pathogenic? Int. J. Parasitol. 28:219-240.
7. Bancroft G. and B. Askonas. 1985. Immunobiology of African Trypanosomiasis in laboratory rodents. In Immunology and Pathogenesis of Trypanosomiasis. I. Tizard, editor. FL:CRC, Boca Raton. 75-102.
8. Askonas B A. 1985. Macrophages as mediators of immunosuppression in murine African trypanosomiasis. Curr. Top. Microbiol. Immunol. 117:119-127.
9. Borowy N. K., J. M. Sternberg, D. Schreiber, C. Nonnengasser and P. Overath. 1990. Suppressive macrophages occurring in murine Trypanosoma brucei infection inhibit T-cell responses in vivo and in vitro. Parasite Immunol. 12:233-246.
10. Kierszenbaum F., S. Muthukkumar, L. A. Beltz and M. B. Sztein. 1991. Suppression by Trypanosoma brucei rhodesiense of the capacities of human T-lymphocytes to express interleukin-2 receptors and proliferate after mitogenic stimulation. Infect. Immun. 59:3518-3522.
11. Wellhausen S. R. and J. M. Mansfield. 1980. Characteristics of the splenic suppressor cell-target cell interaction in experimental African trypanosomiasis. Cell. Immunol. 54:414-424.
12. Sileghem M., A. Dadji, R. Hamers, M. Van de Winkel and P. De Baetselier. 1989. Dual role of macrophages in the suppression of interleukin 2 production and interleukin-2 receptor expression in trypanosome-infected mice. Eur. J. Immunol. 19:829-835.
13. Schleifer K. W. and J. M. Mansfield. 1993. Suppressor macrophages in African trypanosomiasis inhibit T-cell proliferative responses by nitric oxide and prostaglandins. J. Immunol. 151:5492-5503.
14. Mabbott N. A., I. A. Sutherland and J. M. Sternberg. 1995. Suppressor macrophages in Trypanosoma brucei infection: nitric oxide is related to both suppressive activity and lifespan in vivo. Parasite Immunol. 17:143-150.
15. Mabbott N. A., P. S. Coulson, L. E. Smythies, R. A. Wilson and J. M. Sternberg. 1998. African trypanosome infections in mice that lack the interferon-gamma receptor gene: nitric oxide-dependent and -independent suppression of T-cell proliferative responses and the development of anaemia. Immunology 94:476-480.
16. Beschin A., L. Brys, S. Magez, M. Radwanska and P. De Baetselier. 1998. Trypanosoma brucei infection elicits nitric oxide-dependent and nitric oxide-independent suppressive mechanisms. J. Leukoc. Biol. 63:429-439.
17. Dadji A., M. Sileghem, H. Heremans, L. Brys and P. De Baetselier. 1993. Inhibition of T-cell responsiveness during experimental infections with Trypanosoma brucei: active involvement of endogenous gamma interferon. Infect. Immun. 61:3098-3102.
18. Magez S., M. Radwanska, A. Beschin, K. Sekikawa and P. De Baetselier. 1999. Tumor necrosis factor alpha is a key mediator in the regulation of experimental Trypanosoma brucei infections. Infect. Immun. 67:3128-3132.
19. Clayton C. E., D. L. Sacks, B. M. Ogilvie and B. A. Askonas. 1979. Membrane fractions of trypanosomes mimic the immunosuppressive and mitogenic effects of living parasites on the host. Parasite Immunol. 1:241-249.
20. Sacks D. L., G. Bancroft, W. H. Evans and B. A. Askonas. 1982. Incubation of trypanosome-derived mitogenic and immunosuppressive products with peritoneal macrophages allows recovery of biological activities from soluble parasite fractions. Infect. Immun. 36:160-168.
21. Sternberg M. J. and N. A. Mabbott. 1996. Nitric oxide-mediated suppression of T-cell responses during Trypanosoma brucei infection: soluble trypanosome products and interferon-gamma are synergistic inducers of nitric oxide synthase. Eur. J. Immunol. 26:539-543.
22. Olsson T., M. Bakhiet, B. Hojeberg, A. Ljungdahl, C. Edlund, G. Andersson, H. P. Ekre, W. P. Fung-Leung, T. Mak, H. Wigzell, et al. 1993. CD8 is critically involved in lymphocyte activation by a T. brucei brucei-released molecule. Cell 72:715-727.
23. Vaidya T., M. Bakhiet, K. L. Hill, T. Olsson, K. Kristensson and J. E. Donelson. 1997. The gene for a T-lymphocyte triggering factor from African trypanosomes. J. Exp. Med. 186:433-438.
24. Magez S., B. Stijlemans, M. Radwanska, E. Pays, M. A. Ferguson and P. De Baetselier. 1998. The glycosyl-inositol-phosphate and dimyristoylglycerol moieties of the glycosylphosphatidylinositol anchor of the trypanosome variant-specific surface glycoprotein are distinct macrophage-activating factors. J. Immunol. 160:1949-1956.
25. Paulnock D. M. and S. P. Coller. 2001. Analysis of macrophage activation in African trypanosomiasis. J. Leukoc. Biol. 69:685-690.
26. Tachado S. D. and L. Schofield. 1994. Glycosylphosphatidylinositol toxin of Trypanosoma brucei regulates IL-1 alpha and TNF-alpha expression in macrophages by protein tyrosine kinase mediated signal transduction. Biochem. Biophys. Res. Commun. 205:984-991.
27. Kubata B. K., M. Duszenko, Z. Kabututu, M. Rawer, A. Szallies, K. Fujimori, T. Inui, T. Nozaki, K. Yamashita, T. Horii, Y. Urade and O. Hayaishi. 2000. Identification of a Novel Prostaglandin F(2alpha) Synthase in Trypanosoma brucei. J. Exp. Med. 192:1327-1338.
28. Darji A., A. Beschin, M. Sileghem, H. Heremans, L. Brys and P. De Baetselier. 1996. In vitro simulation of immunosuppression caused by Trypanosoma brucei: active involvement of gamma interferon and tumor necrosis factor in the pathway of suppression. Infect. Immun. 64:1937-1943.
29. Jamonneau V., P. Truc, A. Garcia, E. Magnus and P. Buscher. 2000. Preliminary evaluation of LATEX/T b. gambiense and alternative versions of CATT/T b. gambiense for the serodiagnosis of human African trypanosomiasis of a population at risk in Cote d'Ivoire: consideration for mass screening. Acta. Trop. 76:175-183.
30. Lejon V., P. Buscher, E. Magnus, I. Wouters and N. Van Meirvenne. 1998. A semi-quantitative ELISA for detection of Trypanosoma brucei gambiense-specific antibodies in serum and cerebrospinal fluid of sleeping sickness patients. Acta. Trop. 69:151-165.
31. Komba E., M. Odiit, D. B. Mbulamberi, E. C. Chimfwembe and V. M. Nantylya. 1993.

Multicentre evaluation of an antigen-detection ELISA for the diagnosis of Trypanosoma brucei rhodesiense sleeping sickness. Bulletin of the World Health Organisation 70:57-61.
32. Nantulya V. M., F. Doua and S. Molisho. 1992. Diagnosis of Trypanosoma brucei gambiense sleeping sickness using an antigen detection enzyme-linked immunosorbent assay. Transactions of the Royal Society of Tropical Medicine and Hygiene 86:42-45.
33. Ngaira J. M., W. Olaho-Mukani, J. K. Omuse, K. M. Tengekyon, D. Mbwabi, D. Olado and J. N. Njenga. 1992. Evaluation of procyclic agglutination trypanosomiasis test (PATT) for the immunodiagnosis of Trypanosoma brucei rhodesiense sleeping sickness in Kenya. Trop. Med. Parasitol. 43:29-32.
34. Van Meirvenne N. 1992. Diagnosis of human African trypanosomiasis. Ann. Soc. Belg. Med. Trop. 72 Suppl. 1:53-56.
35. Magnus E., T. Vervoort and N. Van Meirvenne. 1978. A card-agglutination test with stained trypanosomes (C.A.T.T.) for the serological diagnosis of T b. gambiense trypanosomiasis. Annales de la Société Belge de Médecine Tropicale 58:169-176.
36. Moser D. R., G. A. Cook, D. E. Ochs, C. P. Bailey, M. R. McKane and J. E. Donelson. 1989. Detection of Trypanosoma congolense and Trypanosoma brucei subspecies by DNA amplification using the polymerase chain reaction. Parasitology 99:57-66.
37. Masiga D. K., A. J. Smyth, P. Hayes, T. J. Bromidge and W. C. Gibson. 1992. Sensitive detection of trypanosomes in tsetse flies by DNA amplification. Int. J. Parasitol. 22:909-918.
38. Dilbeck V., M. Berberof, A. Van Cauwenberge, H. Alexandre and E. Pays. 1999. Characterization of a coiled coil protein present in the basal body of Trypanosoma brucei. J Cell. Sci. 112:4687-4694.
39. Borst P., M. Van Der Ploeg, J. F. Van Hoek, M. Tas and J. James. 1982. On the DNA content and ploidy of trypanosomes. Mol. Biochem. Parasitol. 6:13-23.
40. Verloo D., J. Brandt, N. Van Meirvenne and P. Buscher. 2000. Comparative in vitro isolation of Trypanosoma theileri from cattle in Belgium. Vet. Parasitol. 89:129-132.
41. Herbert W. J. and W. H. R. Lumsden. 1976. Trypanosoma brucei: a rapid “matching” method for estimating the host's parasitaemia. Exp. Parasitol. 40:427-431.
42. Lanham S. M. and D. G. Godfrey. 1970. Isolation of salivarian trypanosomes from man and other mammals using DEAE-cellulose. Exp. Parasitol. 28:521-534.
43. Gross-Bellard M., P. Oudet and P. Chambon. 1973. Isolation of high-molecular-weight DNA from mammalian cells. Eur. J. Immunol. 36:32-38.
44. Xong H. V., L. Vanhamme, M. Chamekh, C. E. Chimfwembe, J. Van Den Abbeele, A. Pays, N. Van Meirvenne, R. Hamers, P. De Baetselier and E. Pays. 1998. A VSG expression site-associated gene confers resistance to human serum in Trypanosoma rhodesiense. Cell 95:839-846.

What is claimed is:

1. An isolated polypeptide having the primary structural information of amino acids 1-553 as set forth in SEQ ID NO:2 or any functional fragment or allelic variant thereof possessing the biological property of having immunomodulating activity.

2. An isolated polynucleotide encoding the isolated polypeptide of claim 1 and set forth in SEQ ID NO:1 or any functional fragment or allelic variant thereof possessing the biological property of encoding immunomodulating activity.

3. The isolated polypeptide of claim 1 wherein said isolated polypeptide shares at least 40% homology with SEQ ID NO:2.

4. The isolated polynucleotide of claim 2 wherein said isolated polynucleotide shares at least 40% homology with SEQ ID NO:1.

5. A vector comprising the isolated polynucleotide of claim 2.

6. The vector of claim 5, wherein said vector is an expression vector, and further comprises a regulatory element.

7. A genetically engineered host cell comprising the vector of claim 6.

8. A vector comprising the isolated polynucleotide of claim 4.

9. The vector of claim 8, wherein said vector is an expression vector, and further comprises a regulatory element.

10. A genetically engineered host cell comprising the vector of claim 9.

11. A recombinant protein comprising a fragment of SEQ ID NO:2 and having immunomodulating activity in a mammal as determined by reducing or suppressing the mammal's immune response after administration.

12. A pharmaceutical composition comprising:
the isolated polypeptide of claim 1 in a pharmaceutically acceptable form suitable for administration to a mammalian subject.

13. A pharmaceutical composition comprising:
the isolated polynucleotide of claim 2 in a pharmaceutically acceptable form suitable for administration to a mammalian subject.

14. A method of suppressing a subject's immune response, the method comprising:
administering to the subject the isolated polypeptide of claim 1.

15. A method of suppressing a subject's immune response, the method comprising:
administering to the subject the isolated polynucleotide of claim 2.

16. A diagnostic assay method for use in detecting the presence of a Trypanozoon infection in a mammal, said diagnostic assay method comprising:
detecting, in a tissue sample of said mammal, means for detecting the presence of a Trypanozoon infection in a mammal, said means selected from the group consisting of an isolated polynucleotide encoding the isolated polypeptide of claim 1 and set forth in SEQ ID NO:1, a functional fragment or allelic variant thereof possessing the biological property of encoding immunomodulating activity, an isolated polypeptide having the primary structural information of amino acids 1-553 as set forth in SEQ ID NO:2, a functional fragment or allelic variant thereof possessing the biological property of having immunomodulating activity, and both the isolated polynucleotide and the isolated polypeptide,
wherein the presence of said means is indicative of a Trypanozoon infection.

17. The diagnostic assay method according to claim 16 wherein said mammal is a human and wherein said Trypanozoon is Trypanosoma brucei gambiense or Trypanosoma brucei rhodesiense and wherein the presence of said Trypanozoon is indicative of sleeping disease in the human.

18. An isolated polypeptide having:
at least 40% homology with SEQ ID NO:4, and
immunomodulating activity in a mammal as determined by reducing or suppressing the mammal's immune response after administration of the isolated polypeptide.


SEQUENCE LISTING (4 sequences)

SEQ ID NO: 1 - DNA, 2826 bp, Trypanosoma brucei
  CDS (256)..(2757)
agtttctgtactatattgagggctcacagagttttctttgaataagatagaaaccgcctaaaggggcagttgatattattatcttgtactgttgcagcgcaaccgctgaagatcaccttttgttttttgaaagaaaaaacacacacacacacacacaagtgcactcaggtgcacgtttcgtagacataattctgtcaagtggaaaaggttgtgtgagccgaacctcgcaacccatagaagagagtgtgaaggaaaatgttgacgctccgtgaactagacccttccgaactggaagatgaagcgcacctgaggcgcttcgaacaatgtcgttgcgaggttatgcgcgtgtccagtagccgtcttctccctgaatgtattgctctgaccacaaaaggcggttttgtacaactcgtcaacgtttccacgggagcgtttcgtgttttccttacacacctgggaaatatgccgttggagtcacaacttcgttgcttttcccttgccccctccctctacgctaccaaaggtgcccactcgctgctatactcacttgcgtactcgaacgaactcctgctggctgatatggagcaaggtactgtgagtgttcttgcaaccttccaaagccgccccagtgttgtgtattgtgatggtgattacattgtgtgcggggagggctctgggcaagtaaccctgtggcggtcacctagtgccacagatggtgatgcgacggttgagtggcgcaggtctgtattcacagacacggtggtatgtattacactacagcgtgcatacgtggtctgttcatctgtagaccaacactcctacgtgctatccctccgtggaggggacatcatcgcaaaacttgcgcaagacccagaggcggcggtttcgctgcagtctgttacaacttcacaactaagcggaggtttcatgttactctgtcttccgaccgtactgactgtttatgctccaccgaacaaagaggttgcagcacccccttgtcccaccgacggtaccgtgtgggactgctcgaattcagtacaactagacacgcagataaactgtttaagttgctgtgttggcctcatagcctgcggaacagcttccggcgttgtaatcctcatgactatggatgaaacaggccgtgaggtggtggagcgagtaaggttcaacgttgggtttgctgtcgtttgtgttcagttgtttgacgacgacaccctggtggttgtaacctcctctggcgatgtatggaagtggaccaccacggacttgcttcctgaggagccagcatgtgatttgagcggtgaggaagaacttccgagcctaatgtgcaaccgcctaccgccacgtctgcggcacccaggccaacacatgaatcaatgggtctggacaaccctcagattgtcaagtaacttcagtgaggttgttaacccaggatatcttaatgagtgcggaaaagaggaggaggaggagattactataatgatggacgatgaatctcatcctctgccgtctcgcgtatcaacccttccctccagacacccagtgaaccttcgcacacattgtcgggaaggtagcgacaataagaatgttggggattaccaggaaatttttgacacaccaaatgaggtgcttgctcatatggatgagtgttctggagatagaagtgatcatatgccgacgtgcgctcgaaccaggtcaccatcgggcacggttacagagtccgaaaccttttctgatgatggcagctcgtgttctagtcgatcgagctcaccgagcagcgtcaaccgtgctgggtatgcgaccgcagatgtagcggccgaccgcatcaccaatggggcaattcagagctcaccacccagcgaacagaaccttcgctcaatcagtcgtgatgctgcgggtgactttgccgttgatgtggatgcgacgctgacctccagtcccgggacttgtgccttgtccacctgtcttggaggccggactgacactgctgacgcagttgctgttgctgcctgcaacgaagtaactgagcgtggaggtgaggtgtgctctcccacaaggtatacactgacgcacaccctggacaaggtgttacgtgctgttgatgtagtcccgccgttaacgatcaaggggttgtgcgcgggaaggcgtatggacccgcgcaaggccaaacttattgctgagaccgactgccatgaagagccagccccgggaccggagaagcaaagttcacaaatgttgatggattatcgtgaagggctggaggacacgccctttgactatgcggcgtacgctggggcccacccactaaagaccgaggctcttaaatacagataccccgtgcgaatgccggtgtacagtacacgcgctcaactgttcactccttccacggaactgcgcgtgaccaacatcaaaggaaatagcgaagaaaacaatatggaggtcaatcggaagaaagaaagccgacaagaccacaatgatcagcaagagaatgtcacgaattttaccattgccgatgatctcatgaacgccacgtacggaagactcacacggcgaaaagatcccctgcttgaggaagagcgacagcttgggggacccgatattcgtcgcactcttgcggtggattactgtgtgggaccgtggatcctaatggaactgtgctttttcaggaatttcgcgtgcagcccagtcctcctggtctacttcttttaccaatcccactccccccgacgcccacagtcttttaatatgattccatagagtggaataagatgaacgtgcatcgtttgatgcggtttgttgtcatttttctttttttaaaaaaaaaaa

SEQ ID NO: 2 - protein, 833 aa, Trypanosoma brucei
MLTLRELDPSELEDEAHLRRFEQCRCEVMRVSSSRLLPECIALTTKGGFVQLVNVSTGAFRVFLTHLGNMPLESQLRCFSLAPSLYATKGAHSLLYSLAYSNELLLADMEQGTVSVLATFQSRPSVVYCDGDYIVCGEGSGQVTLWRSPSATDGDATVEWRRSVFTDTVVCITLQRAYVVCSSVDQHSYVLSLRGGDIIAKLAQDPEAAVSLQSVTTSQLSGGFMLLCLPTVLTVYAPPNKEVAAPPCPTDGTVWDCSNSVQLDTQINCLSCCVGLIACGTASGVVILMTMDETGREVVERVRFNVGFAVVCVQLFDDDTLVVVTSSGDVWKWTTTDLLPEEPACDLSGEEELPSLMCNRLPPRLRHPGQHMNQWVWTTLRLSSNFSEVVNPGYLNECGKEEEEEITIMMDDESHPLPSRVSTLPSRHPVNLRTHCREGSDNKNVGDYQEIFDTPNEVLAHMDECSGDRSDHMPTCARTRSPSGTVTESETFSDDGSSCSSRSSSPSSVNRAGYATADVAADRITNGAIQSSPPSEQNLRSISRDAAGDFAVDVDATLTSSPGTCALSTCLGGRTDTADAVAVAACNEVTERGGEVCSPTRYTLTHTLDKVLRAVDVVPPLTIKGLCAGRRMDPRKAKLIAETDCHEEPAPGPEKQSSQMLMDYREGLEDTPFDYAAYAGAHPLKTEALKYRYPVRMPVYSTRAQLFTPSTELRVTNIKGNSEENNMEVNRKKESRQDHNDQQENVTNFTIADDLMNATYGRLTRRKDPLLEEERQLGGPDIRRTLAVDYCVGPWILMELCFFRNFACSPVLLVYFFYQSHSPRRPQSFNMIP

SEQ ID NO: 3 - DNA, 1665 bp, Trypanosoma brucei
  CDS (1)..(1665)
ggaacagcttccggcgttgtaatcctcatgactatggatgaaacaggccgtgaggtggtggagcgagtaaggttcaacgttgggtttgctgtcgtttgtgttcagttgtttgacgacgacaccctggtggttgtaacctcctctggcgatgtatggaagtggaccaccacggacttgcttcctgaggagccagcatgtgatttgagcggtgaggaagaacttccgagcctaatgtgcaaccgcctaccgccacgtctgcggcacccaggccaacacatgaatcaatgggtctggacaaccctcagattgtcaagtaacttcagtgaggttgttaacccaggatatcttaatgagtgcggaaaagaggaggaggaggagattactataatgatggacgatgaatctcatcctctgccgtctcgcgtatcaacccttccctccagacacccagtgaaccttcgcacacattgtcgggaaggtagcgacaataagaatgttggggattaccaggaaatttttgacacaccaaatgaggtgcttgctcatatggatgagtgttctggagatagaagtgatcatatgccgacgtgcgctcgaaccaggtcaccatcgggcacggttacagagtccgaaaccttttctgatgatggcagctcgtgttctagtcgatcgagctcaccgagcagcgtcaaccgtgctgggtatgcgaccgcagatgtagcggccgaccgcatcaccaatggggcaattcagagctcaccacccagcgaacagaaccttcgctcaatcagtcgtgatgctgcgggtgactttgccgttgatgtggatgcgacgctgacctccagtcccgggacttgtgccttgtccacctgtcttggaggccggactgacactgctgacgcagttgctgttgctgcctgcaacgaagtaactgagcgtggaggtgaggtgtgctctcccacaaggtatacactgacgcacaccctggacaaggtgttacgtgctgttgatgtagtcccgccgttaacgatcaaggggttgtgcgcgggaaggcgtatggacccgcgcaaggccaaacttattgctgagaccgactgccatgaagagccagccccgggaccggagaagcaaagttcacaaatgttgatggattatcgtgaagggctggaggacacgccctttgactatgcggcgtacgctggggcccacccactaaagaccgaggctcttaaatacagataccccgtgcgaatgccggtgtacagtacacgcgctcaactgttcactccttccacggaactgcgcgtgaccaacatcaaaggaaatagcgaagaaaacaatatggaggtcaatcggaagaaagaaagccgacaagaccacaatgatcagcaagagaatgtcacgaattttaccattgccgatgatctcatgaacgccacgtacggaagactcacacggcgaaaagatcccctgcttgaggaagagcgacagcttgggggacccgatattcgtcgcactcttgcggtggattactgtgtgggaccgtggatcctaatggaactgtgctttttcaggaatttcgcgtgcagcccagtcctcctggtctacttcttttaccaatcccactccccccgacgcccacagtcttttaatatgattccatag

SEQ ID NO: 4 - protein, 554 aa, Trypanosoma brucei
GTASGVVILMTMDETGREVVERVRFNVGFAVVCVQLFDDDTLVVVTSSGDVWKWTTTDLLPEEPACDLSGEEELPSLMCNRLPPRLRHPGQHMNQWVWTTLRLSSNFSEVVNPGYLNECGKEEEEEITIMMDDESHPLPSRVSTLPSRHPVNLRTHCREGSDNKNVGDYQEIFDTPNEVLAHMDECSGDRSDHMPTCARTRSPSGTVTESETFSDDGSSCSSRSSSPSSVNRAGYATADVAADRITNGAIQSSPPSEQNLRSISRDAAGDFAVDVDATLTSSPGTCALSTCLGGRTDTADAVAVAACNEVTERGGEVCSPTRYTLTHTLDKVLRAVDVVPPLTIKGLCAGRRMDPRKAKLIAETDCHEEPAPGPEKQSSQMLMDYREGLEDTPFDYAAYAGAHPLKTEALKYRYPVRMPVYSTRAQLFTPSTELRVTNIKGNSEENNMEVNRKKESRQDHNDQQENVTNFTIADDLMNATYGRLTRRKDPLLEEERQLGGPDIRRTLAVDYCVGPWILMELCFFRNFACSPVLLVYFFYQSHSPRRPQSFNMIP